SEQUENCE LISTING (5 sequences)

SEQ ID NO: 1 - DNA, 19 bp, Artificial Sequence
  Oligonucleotide-Primer
gcttgcattccctccagga

SEQ ID NO: 2 - DNA, 21 bp, Artificial Sequence
  Oligonucleotide-Primer
tggaccctctgcagctgatgt

SEQ ID NO: 3 - DNA, 27 bp, Artificial Sequence
  Oligonucleotide-Primer
ggatccatgtctaagggacctgcagtt

SEQ ID NO: 4 - DNA, 30 bp, Artificial Sequence
  Oligonucleotide-Primer
gaattcgacttaatcgacctcttcaatggt

SEQ ID NO: 5 - protein, 14 aa, Artificial Sequence
  Peptide
TKDNNLLGRFELSG

(19) United States
(12) Patent Application Publication
(10) Pub. No.: US 2010/0034772 A1
(43) Pub. Date: Feb. 11, 2010

(54) COMPOSITIONS AND METHODS FOR IMMUNOTHERAPY

(76) Inventors: Ralf Dressel, Gottingen (DE); Leslie Elsner, Gottingen (DE)

Correspondence Address: COOPER & DUNHAM, LLP, 30 Rockefeller Plaza, 20th Floor, NEW YORK, NY, 10112, US

(21) Appl. No.: 12/312,321

(22) PCT Filed: Nov. 14, 2007
(86) PCT No.: PCT/EP2007/009857

§ 371 Date: May 5, 2009

(30) Foreign Application Priority Data
Nov. 14, 2006 (EP) 06023692.4

(51) Int. Cl. A61K 38/20; A61K 35/12; A61K 38/10; A61K 31/7088

(52) U.S. Cl. 424/85.2; 424/93.7; 514/14; 514/44 R

(57) ABSTRACT

The present invention relates to the technical field of immunology and the treatment of diseases mediated and/or indicated by the presence of a ligand of NK receptor NKG2D, such as major histocompatibility complex class I chain related (MIC) A and B molecules on the cell surface of diseased tissue or cells. In particular, the present invention makes use of the surprising finding that the stress-inducible endogenous danger signals HSP70 and MICA/B synergistically activate NK cells against tumors. Methods and compositions for the prevention and treatment of infectious diseases, primary and in particular metastatic neoplastic diseases, including, but not limited to human sarcomas, carcinomas and melanomas are provided.

### FIELD OF THE INVENTION

The present invention generally concerns the field of immunotherapy. More specifically, the present invention relates to methods and compositions for the prevention and treatment of infectious diseases, primary and, especially, metastatic neoplastic diseases, including, but not limited to human sarcomas, carcinomas and melanomas. In accordance with the present invention, the practice of the prevention and treatment of infectious diseases and cancer is mediated and/or indicated by the presence of certain ligands on the cell surface of diseased tissue or cells, which make them susceptible to immunotherapy, in particular natural killer (NK) cell based therapy.

### BACKGROUND OF THE INVENTION

The era of tumor immunology began with experiments by Prehn and Main, who showed that antigens on the methylcholanthrene (MCA)-induced sarcomas were tumor specific in that transplantation assays could not detect these antigens in normal tissue of the mice (Prehn et al., J. Natl. Cancer Inst. 1 (1957), 769 778). This notion was confirmed by further experiments demonstrating that tumor specific resistance against MCA-induced tumors can be elicited in the autochthonous host, that is, the mouse in which the tumor originated. Radiation is frequently used in cancer therapy, either as a single regimen or in combination with cytostatic drugs as radiochemotherapy. However, irradiation-resistant tumor clones are limiting the therapeutic efficiency.

The stress-inducible heat shock protein (HSP) 70 is a molecular chaperone which is well known to protect cells against apoptosis (1). Overexpression of HSP70 has been described in various tumors and was found to be associated with enhanced tumorigenicity and resistance to therapy (2). In accord with these findings, experimental downregulation of HSP70 in tumor cells was reported to enhance tumor regression in animal models (3-5). However, in several other animal models contrary observations were made. HSP70 expression was found to be associated with tumor regression (6-9). In these cases, HSP70 appeared to augment the immunogenicity of tumors. In numerous studies HSP70 has been shown to activate innate and adaptive immune reactions (10, 11). HSP70 chaperones antigenic peptides and channels them in a receptor-mediated manner into the major histocompatibility complex (MHC) class I presentation pathway of professional antigen presenting cells, which then prime peptide-specific CTL. Therefore, HSP70 preparations from tumors can be used for tumor-specific vaccination (10, 11). HSP70 also elicits the release of pro-inflammatory cytokines from innate immune cells and augments the expression of co-stimulatory molecules (10, 11). Furthermore, HSP70 has been shown to activate natural killer (NK) cells to kill specifically tumor cells which express HSP70 at the cell surface (12). Due to these features, HSP70 has been viewed as an endogenous adjuvant and immunological danger signal (13, 14).

### SUMMARY OF THE INVENTION

The present invention relates to the technical field of immunology and the treatment of diseases mediated and/or indicated by the presence of a ligand of NK receptor NKG2D, such as major histocompatibility complex class I chain related (MIC) A and B molecules on the cell surface of diseased tissue or cells. In particular, the present invention makes use of the surprising finding that the stress-inducible endogenous danger signals HSP70 and MICA/B synergistically activate NK cells against tumors.

Since HSP70 is known to be anti-apoptotic but can also elicit a CTL response, one question posed was as to whether HSP70 protects tumor cells against apoptosis mediated by CTL. In the model of the human melanoma cell line Ge, it could be shown previously that constitutive overexpression of the MHC-linked stress-inducible HSP70 does not protect against apoptosis mediated by CTL in the granule exocytosis pathway (15). Acute HSP70 overexpression can even increase the susceptibility against CTL in vitro (15, 16). The immune system appears to be able to kill target cells undergoing an otherwise protective stress response.

The object of the present invention was to determine the effect of HSP70 expression on the susceptibility of Ge melanoma cells to adoptively transferred CTL in vivo. However, in severe combined immunodeficient (SCID) mice, lacking B and T lymphocytes, the growth of HSP70 overexpressing tumors was reduced compared to control tumors even before any adoptive immunotherapy. More impressively, invasive growth and regional metastases were only observed in animals bearing non-HSP70 overexpressing control tumors. In accordance with present invention it could surprisingly be shown that the stress-inducible danger signal HSP70 activated mouse NK cells in SCID mice, as well as human NK cells in vitro which recognized a second stress-inducible danger signal on tumor cells—the MHC class I chain-related (MIC) A and B molecules. MICA and MICB genes are encoded within the MHC, are stress inducible, and are expressed in a restricted manner in intestinal epithelial cells and in tumors (17). MICA and MICB are ligands for the activating NK receptor NKG2D (18). Both endogenous stress-inducible danger signals, HSP70 and MICA/B, synergistically elicited a NK cell-mediated immune response against tumor cells. In the animal model this two danger signals-driven innate immune response was able to reduce the growth of primary tumors and to suppress metastases.

In a first aspect, the present invention relates to the use of NK cells, preferably activated NK cells or an activator of NK cells for the preparation of a pharmaceutical composition for the treatment of a disease in a subject, wherein said disease involves cells which express or are induced to express a ligand for NK cell receptor NKG2D on the cell surface. Preferably, said ligand is MHC class I chain-related (MIC) A or B and said NK cells are activated prior to administration to the patient or are designed to be administered in conjunction with an activator of NK cells, for example HSP70 or a peptide derived thereof.

The use of the compounds in accordance with the present invention may be accompanied by the use of further therapeutic agents such as interleukins, interferons or other anti-cancer drugs. Furthermore, the pharmaceutical composition of the present invention may comprise or can be designed to be administered in conjunction with an inducer of the expression of said ligands on the cell surface, for example a histone deacetylase inhibitor such as trichostatin A or suberoylanilide hydroxyamic acid (SAHA).

It is a further object of the present invention to provide the use of a ligand of NKG2D, a nucleic acid molecule encoding said ligand, or inducer of the expression of said ligand on the cell surface for the preparation of a pharmaceutical composition for inducing and/or enhancing an immune response, i.e. cytolytic attack of NK cells against undesired cells. Said pharmaceutical composition may further comprise an agent for inducing or enhancing the expression of HSP70 on the cell surface of said undesired cell.

It is a further object of the present invention to provide the use of NK cells or an activator of NK cells for the preparation of a pharmaceutical composition for the treatment of a tumor or infectious disease in a subject, which disease has been positively tested to be due to cells expressing a ligand of NKG2D on the cell surface. Said NK cells may be activated prior to administration to the subject or are designed to be administered in conjunction with an activator of NK cells. Preferably, said activator comprises a peptide of Hsp70. Furthermore, if the diseased cells substantially lack expression of HSP70 on the cell surface, said cells may be induced to express HSP70 on the cell surface as well.

In addition, the present invention concerns a combination preparation comprising a ligand of NKG2D, a nucleic acid molecule encoding said ligand, or inducer of the expression of said ligand on the cell surface in combination with an activator of NK cells, for example HSP70 or a peptide derived thereof and/or an agent capable of inducing expression of HSP70 on the cell surface, useful for the targeting and/or treatment of a tumor or an infectious disease.

Naturally, the pharmaceutical compositions can be administered in combination with other drugs such as chemotherapeutic agents. Suitable chemotherapeutic agents are known to those skilled in the art and include anthracyclines (e.g. daunomycin and doxorubicin), taxol, methotrexate, vindesine, neocarzinostatin, cis-platinum, chlorambucil, cytosine arabinoside, 5-fluorouridine, melphalan, ricin and calicheamicin, the choice of which may be dependent on the disease intended to be treated.

While most embodiments are described in context with medical uses it is to be understood that the scope of the present invention and thus of the appended claims encompass cell/tissue culture methods and animal testing for research purposes only as well.

### BRIEF DESCRIPTION OF THE DRAWINGS

FIG. 1:

Tumors derived from Hsp70 overexpressing melanoma cells grow slower and do not give rise to regional metastases. Ge-Hsp70 or Ge-con cells were injected subcutaneously into the flank of SCID mice (1×10⁶ cells in PBS/animal). Mean of tumor size±SD for SCID mice in which tumor growth was observed.

FIG. 2:

Proliferation and apoptosis rates of Ge-Hsp70 and Ge-con cells do not differ in vitro. A, proliferation of Ge-con (Ge-TCR-C, Ge-GFP-B) and Ge-Hsp70 (Ge-Hsp70-A, Ge-Hsp70-C) cells in vitro was assayed by [3H]-thymidine incorporation. Mean of cpm±SD of triplicates are shown of an experiment which is representative for 3 independent assays. B, the melanoma cells were cultured for 24 h in a hypoxic atmosphere or in glucose free medium. The percentage of apoptotic cells (% of cells in the sub-G1 peak of DNA histograms) was determined by flow cytometry before (pre) and 2, 24, and 48 h after treatment. Mean of apoptotic cells+SD of 4 independent experiments is shown.

FIG. 3:

NK cells are responsible for the reduced growth of Hsp70 overexpressing melanoma cells in SCID mice. A, mean of the percentage of NK cells in the spleen+SD, detected by flow cytometry using an anti-DX5 mAb is not markedly different between SCID mice bearing Ge-con or Ge-Hsp70 tumors or animals which rejected the tumor cells (no tumors). B, mean of specific lysis±SD of triplicates of YAC-1 target cells by splenocytes derived from 3 SCID mice bearing Ge-con tumors and 3 SCID mice bearing Ge-Hsp70 tumors at different effector target ratios. The experiment shown here is representative of 5 independent assays. C, Ge-Hsp70 or Ge-con cells were injected subcutaneously into the flank of SCID/beige mice (1×10⁶ cells in PBS/animal). Mean of tumor size +SD for animals in which tumor growth was observed.

FIG. 4:

Tumors which do not express HSP70 at the cell surface release HSP70 in exosomes. Exosomes were prepared from Ge-Hsp70 and Ge-con cells. 10 μg of exosomal proteins were separated by SDS-PAGE and analyzed for the presence of HSP70 by an immunoblot using the mAb C92 which is specific for the inducible HSP70. An anti-Rab4 Ab was used as loading control for the exosomal proteins.

FIG. 5:

LAK cells readily lyse MICA transfected target cells. A, flow cytometric analysis of MICA and HSP70 expression on MICA-transfected L-MICA and parental L cells. MICA cell surface expression is shown by staining with an anti-rhesus MICA antiserum, two monoclonal antibodies against human MICA and MICB, and by binding of a recombinant human NKG2D-Fc fusion protein. Staining with the respective primary reagent (black line) and FITC-labeled secondary reagent only (dashed line) is shown together with unstained cells (dotted line). For the anti-rhesus MICA antiserum a staining with the preimmune serum plus secondary antibody (dashed line) is shown as control. The results are representative for more than 3 independent experiments. Hybridoma supernatants from clones BAMO3, and IIIC1, both reacting with human MICA and MICB, were used. An anti-rhesus MICA antiserum was generated by immunizing FVB/N mice with MICA expressing lymphocytes from transgenic FVB/N mice containing the cosmid A158 that carries the rhesus macaque MICA gene. B, mean of specific lysis+SD of triplicates of K562, L and L-MICA target cells by freshly isolated PBMC or LAK cells from the same donor stimulated in vitro for 4 days by IL-2 (100 U/ml). C, mean of relative lysis+SD of K562, L-MICA, and L target cells by PBMC or LAK effector cells as determined in 8 independent experiments. The percentage of lysis of K562 cells by PBMC at the highest effector target ratio (100:1) was adjusted to 100 % in each test and the relative lysis of the various target cells by PBMC and LAK cells at different effector:target ratios was calculated. L-MICA cells were killed significantly better by human LAK cells than by unstimulated PBMC (p=0.0003) whereas the classical NK target cell line K562, was lysed more readily by PBMC (p=0.0121) as analyzed by ANOVA separately for each cell type.

FIG. 6:

MICA/B expression is induced in tumors which do not express HSP70 at the cell surface. A, flow cytometric analysis of HSP70 cell surface expression on cells isolated from Ge-con and Ge-Hsp70-derived tumors. The percentage of positive cells is given. The experiment shown is representative for 8 tumors from SCID and 10 from SCID/beige mice. K562 cells served as positive control. B, mean of mRNA expression+SD of MICA, MICB, human HSP70-2 and rat Hsp70-1 determined as ratio to β-actin by Northern blot analysis and densitometry. We analysed 18 Ge-con and 21 Ge-Hsp70 tumors from SCID mice and 16 Ge-con and 14 Ge-Hsp70 tumors from SCID/beige mice. The data for the cell lines in vitro were obtained from 7 to 12 individual experiments. C, flow cytometric analysis of MICA/B (mAb BAMO-1), HSP70 (mAb RPN 1197) and NKG2D ligands cell surface expression on cells isolated from Ge-con-derived tumors. More than 95 % of the gated cells were human melanoma cells which were positive for human MHC class I molecules (mAb W6/32). The upper and the lower panel were derived from different individual tumors.

FIG. 7:

LAK cells are stimulated by HSP70 and the HSP70-derived peptide TKD to kill MICA expressing target cells. A, mean of specific lysis±SD of triplicates of L-MICA (closed symbols) or L (open symbols) target cells by PBMC stimulated in vitro for 7 days with IL-2 only (100 U/ml) or IL-2 plus recombinant HSP70 (2 μg/ml) or HSC70 (2 μg/ml). The experiment shown is representative for 3 independent experiments. B, Mean of relative lysis+SD of L-MICA and L targets by LAK cells stimulated for 7 days with IL-2 (100 U/ml), IL-2 plus HSP70 (2 μg/ml), or IL-2 plus LPS (10 ng/ml), as determined in 6 independent experiments. The percentage of lysis of L-MICA cells by IL-2 stimulated PBMC at the highest effector target ratio (50:1) was adjusted to 100% in each test and the relative lysis of the target cells by various effector cells at different effector:target ratios was calculated. C, mean of relative lysis+SD of L-MICA and L targets by LAK cells stimulated for 7 days with IL-2 (100 U/ml), or IL-2 plus TKD (2 μg/ml), as determined in 6 independent experiments.

FIG. 8:

NK cells are stimulated by the HSP70-derived peptide TKD to kill MICA expressing target cells. A, flow cytometric analysis of PBMC (before MACS separation) and NK cell enriched (NK⁺) as well as NK cell depleted (NK) cell populations. The mean of percentage of marker positive cells+SD of 7 independent experiments is given. B, mean of specific lysis±SD of triplicates of L-MICA and L target cells by isolated NK cells (NK⁺) or NK cell depleted PBMC (NK⁻) stimulated in vitro for 5 days with IL-2 (100 U/ml) or IL-2 plus TKD (2 μg/ml). The experiment shown is representative for 3 experiments with NK and 7 for NK⁺ cells as effector cells. C, mean of relative lysis+SD of L-MICA and L targets by NK cells stimulated for 5 days with IL-2 (100 U/ml), or IL-2 plus TKD (2 μg/ml), as determined in 7 independent experiments.

FIG. 9:

Induction of MICA/B on Ge-con and Ge-Hsp70 melanoma cells increases susceptibility towards LAK cell-mediated cytotoxicity. A, flow cytometric analysis was performed of Ge-con cells for cell surface expression of HSP70 (mAb RPN 1197), MICA/MICB (mAb BAMO1), and NKG2D ligands (human or mouse NKG2D-IgG-Fc fusion protein). Cells were either cultured under standard conditions (co) or exposed to 10 μM SAHA for 20 h before the test. Mean+SD of cells showing cell surface expression of MICA/B, ligands of human NKG2D, and HSP70 and mean intensity of fluorescence (MIF)+SD for Ge-con and Ge-HSP70 cells as obtained in 9 independent experiments. B, mean of relative lysis+SD of Ge-con and Ge-Hsp70 cells by PBMC stimulated for 4 days with IL-2 (100 U/ml). The target cells were either cultured under standard conditions (co) or exposed to 10 μM SAHA for 20 h before the test. The percentage of lysis of the respective cells by the LAK cells at the highest effector target ratio (100:1) was adjusted to 100% in each test (n=4) and the relative lysis of the target cells by various effector cells at different effector:target ratios was calculated. SAHA-treated cells were lysed signifcantly better than untreated cells (p=0.0030, three-way ANOVA for treatment, target cell type and effector:target ratios 100:1 and 50:1). As the target cell type was also important in combination with SAHA treatment (p=0.0006) a stratified ANOVA by cell type was carried out confirming this result for both target cell lines (Ge-TCR-C: p=0.0075 and Ge-Hsp70-A: p=0.0045).

FIG. 10:

The combination of HSP70 peptide TKD treatment of NK cells and MICA/B induction on Ge-con target cells synergistically augment killing. This might be due to expression of increased amounts of granzyme B after stimulation with TKD. A, mean of specific lysis±SD of triplicates of Ge-con target cells by NK cells cultured for 5 days with or without IL-2 (100 U/ml) in combination or not with TKD (2 μg/ml). The target cells were either cultured under standard conditions (co) or exposed to 10 μM SAHA for 20 h before the test. B, mean of relative lysis+SD of Ge-con cells by NK cells stimulated for 5 days with IL-2 (100 U/ml) in combination or not with TKD (2 μg/ml) as determined in 4 independent experiments. The target cells were either cultured under standard conditions (co) or exposed to 10 μM SAHA for 20 h before the test. C, flow cytometric analysis of MACS enriched NK cells before (NK⁺) and after 5 d culture with IL-2 (100 U/ml) or IL-2 (100 U/ml) plus TKD (2 μg/ml). The mean of percentage of marker positive cells+SD of 7 independent experiments is given. D, The mean intensity of fluorescence (MFI)+SD for granzyme B of 5 independent experiments is shown as determined by flow cytometry after intracellular staining.

### DETAILED DESCRIPTION OF THE INVENTION

The present invention relates to methods and compositions for research, prevention and treatment of primary and metastatic neoplastic diseases and infectious diseases and for eliciting an immune response in a mammal, particularly human individual to a desired target cell. In particular, according to one aspect of the invention, the use of NK cells or an activator of NK cells for the preparation of a pharmaceutical composition for the treatment of a disease in a subject is provided, wherein said disease involves cells which express or are induced to express a ligand for NK cell receptor NKG2D on the cell surface.

The present invention is based on the observation that stress-inducible heat shock protein (HSP) 70, known to function as an endogenous danger signal which can increase the immunogenicity of tumors and induce CTL responses, is also capable of activating natural killer (NK) cells which recognize the stress-inducible major histocompatibility complex class I chain related (MIC) A and B molecules on tumor cells by the activating NK receptor NKG2D. As demonstrated in the appended Examples, size of tumors and rate of metastases derived from HSP70 overexpressing human melanoma cells were reduced in T and B cell deficient SCID mice but not in SCID/beige mice which lack additionally functional NK cells. A counter selection was observed against the expression of MICA/B in HSP70 overexpressing tumors in SCID but not in SCID/beige mice. Tumor-derived HSP70 was able to activate NK cells in SCID mice which appeared to kill MICA/B expressing tumor cells. In accord with the observations in the animal model, full-length HSP70 and a HSP70-derived peptide were able to activate in vitro human NK cells to kill MICA transfected target cells and melanoma cells in which MICA/B expression was induced by pharmacological means. Thus, the synergistic activity of two stress-inducible danger signals, HSP70 and MICA/B, leads to enhanced activation of NK cells resulting in reduced tumor growth and suppression of metastases.

Since NK cells and HSP70, respectively, are known to be involved in combating infectious diseases as well, the present invention can be practice on tumor and infectious diseases. Hence, in principle any target cell may rendered amenable for immunotherapy in accordance with present invention by making the desired or undesired cell expressing the NKG2D ligand on its cell surface.

“NK cells” as used herein refer to lymphocytes preferably of human origin, which typically have CD16 and/or NCAM and/or CD56 molecules expressed as cell surface markers but which do not express CD3. The NK cells refer to cells present in vivo in a mammal or in vitro in the form of a purified population of cells. “NK cell activating agent” or “activator of NK cells” as used herein refers to agents which are able to enhance or increase cytolytic activity of resting (or untreated) NK cells in mammalian cancer cells or virus-infected cells. Such agents include but are not limited to, agents which activate one or more Toll receptors, such as Granzyme A or Granzyme B, various interleukins, such as IL-2, IL-12 IL-15, and interferons such as IFN-alpha, IFN-beta.

Ligands of/for NK cell receptor NKG2D are well known to the person skilled in the art and comprise, e.g., MICA, MICB, and members of the UL16-binding protein family (ULBP) 1-4; see for example Friese et al., Cancer Research 63 (2003), 8996-9006. ULBPs, human ligands of the NKG2D receptor have also been characterized and described by Sutherland et al., Blood 108 (2006), 1313-1319. In addition, international application WO2005/080426 describes a novel member of the RAET1/ULBP family of proteins (RAET1G) to bind the UL16 and NKG2D receptors with high affinity.

Furthermore, artificial ligands such as monoclonal antibodies that bind to NKG2D extracellular domains may be used; see, for example, international application WO02/068615 which describes inter alia the use of anti-NKG2D antibodies and ligand derivatives for stimulating a cell expressing an NKG2D receptor, including artificially engineered cell populations. In this context, it is to be understood that in accordance with the present invention target cells may be genetically engineered to express a natural or artificial ligand of NKG2D either anew or at an induced/enhanced level so as to render the given cell susceptible to the treatments and uses of the present invention.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

Furthermore, the term “subject” as employed herein relates to animals in need of immunotherapy, e.g. amelioration, treatment and/or prevention of a neoplastic or infectious disease. Most preferably, said subject is a human.

The pharmaceutical compositions of the present invention can be formulated according to methods well known in the art; see for example Remington's Pharmaceutical Sciences. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use of the pharmaceutical composition. Furthermore, the pharmaceutical composition may also be formulated as a vaccine, for example, if the pharmaceutical composition of the invention comprises a ligand of NKG2D for passive immunization.

In addition, co-administration or sequential administration of other agents may be desirable. A therapeutically effective dose or amount refers to that amount of the active ingredient sufficient to ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The pharmaceutical compositions in accordance with the present invention can be used for the treatment of diseases related to a disorder of the immune response, preferably for the treatment of infectious diseases, sepsis, diabetes or for the treatment of tumors. Preferably, the tumor to be treated is selected from the group consisting of gynecological tumors such as prostate tumor, glioblastoma, medulloblastoma, astrocytoma, primitive neuroectoderma, brain stem glioma cancers, colon carcinoma, bronchial carcinoma, squamous carcinoma, sarcoma, melanoma, carcinoma of colon, cervix or pancreas, carcinoma in the head/neck, T cell lymphoma, B cell lymphoma, mesothelioma, leukemia, melanoma, gynecological tumors such as prostate, and meningeoma.

As mentioned above and demonstrated in the appended Examples, the concept underlying the present invention has been validated using MHC class I chain-related (MIC) A and B, respectively, as the ligand of NKG2D. Therefore, MICA/B or functional equivalent molecules are the preferred ligands to be used in accordance with the present invention.

The nucleotide and amino acid sequences of Human MHC class I polypeptide-related sequence A (MICA) are described in the prior art and can be easily retrieved from public databases such GenBank; see for example GenBank accession no. XM_{—}001124652 (NM_{—}000247) and the publication by, e.g., Vernet et al., Immunogenetics 38 (1993), 47-53. Similarly, the nucleotide and amino acid sequences of MICB are publicly available; see for example GenBank accession nos. NM_{—}005931 and BC044218 as well as publications by, e.g., Bahram et al. in Proc. Natl. Acad. Sci. U.S.A. 91 (1994), 6259-6263; Immunogenetics 45 (1996), 161-162; and Immunogenetics 43 (1996), 230-233.

Means and methods for determining the presence of MIC polypeptide, either MICA or MICB or both, in a sample from a subject are well known to the person skilled in the art; see for example international WO03/089616. These methods may be implemented to the medical uses of the present invention, for example for cancer therapy involving detecting cancer in a subject by assaying for MIC polypeptide on the diseased cell and then administering cancer therapy described herein.

Said NK cells may be activated prior to administration to the patient or can be designed to be administered in conjunction with an activator of NK cells. Preferably, said activator comprises a peptide of Hsp70, which has been shown in the appended Examples to be most effective for this purpose. Furthermore, methods of activating NK cells by the use of Hsp70 protein or fragments thereof and the medical applications of the products so obtained, such as pharmaceuticals, medicinal products or medicinal adjuvants containing Hsp70 protein or fragments thereof or activated NK cells are described in international application WO99/49881. Furthermore, immunostimulatory peptides derived from Hsp70 protein and the use of such peptides for the stimulation of NK cell activity are disclosed in international application WO02/22656. In a particularly preferred embodiment of the present invention the NK activating agent is a HSP70 peptide substantially consisting of the amino acid sequence TKDNNLLGRFELSG (SEQ ID NO: 5); see also the appended Examples.

The pharmaceutical compositions of the present invention can be administered in conjunction with a further immunostimulatory agent, preferably an interleukin such as IL-2 and/or IL-15. As shown in the Examples, the use of interleukin IL-2 is advantageous for the activation of the immune cells for the treatment of malignant cells and therefore is preferably used.

Experiments performed in the scope of the present invention further revealed that upon pharmacologic induction of the expression of the ligand of NKG2D, i.e. MICA/B in Ge melanoma cells these can become a target for HSP70-activated human NK cells; see appended Example 9. Thus, the pharmaceutical composition of the present invention preferably comprises or is designed to be administered in conjunction with an inducer of the expression of the ligand of NKG2D on the cell surface. Preferably, said inducer is a histone deacetylase inhibitor, for example trichostatin A or suberoylanilide hydroxyamic acid (SAHA) used in the Examples. However, other inducers of the expression of the ligand of NKG2D on the cell surface may be used as well, for example sodium valproate (Armeanu et al., Cancer Res. 65 (2005), 6321), ionizing radiation, mitomycin C, hydroxyurea, 5-fluoruracil, aphidicolin, chloroquine (Gasser et al. Nature 463 (2005), 1186).

Furthermore, ligands of NKG2D can be provided as a target recognition structure for the cytolytic attack mediated by NK cells. Thus, in a further embodiment the present invention relates to the use of a ligand of NKG2D, a nucleic acid molecule encoding said ligand, or an inducer of the expression of said ligand on the cell surface for the preparation of a pharmaceutical composition for inducing and/or enhancing cytolytic attack of NK cells against undesired cells. Said NK cells may be activated prior to administration to the subject or are designed to be administered in conjunction with an activator of NK cells. Preferably, said activator comprises a peptide of Hsp70; see supra. Furthermore, if the diseased cells substantially lack expression of HSP70 on the cell surface, said cells may be induced to express HSP70 on the cell surface as well.

Thus, said pharmaceutical composition may further comprise an agent for inducing or enhancing the expression of HSP70 on the cell surface of said undesired cell.

Furthermore, said pharmaceutical composition may additionally contain at least one compound which enhances an immune response or is designed to be administered in conjunction with such compound; see also supra. Preferably, said undesired cells to be treated are tumor cells or infected cells. As already described before, said pharmaceutical composition can be designed to be administered prior, during or after exposure of the undesired cell to an NK activating agent.

As mentioned hereinbefore, ligands of NKG2D expressed on the cell surface of a diseased cell or tissue provide novel recognition structures for the efficacious targeting of the cytolytic attack of NK cells. While a synergistic effect of the presence of HSP70 and the ligand, i.e. MICA/B on tumor cells for cytolytic attack has been observed and thus cells and tissue displaying the combined expression/presence of those classes of molecule on the cell surface are preferred target cells, the presence of a ligand of NKG2D alone is already sufficient to render the (un)desired cell susceptible to the cytolytic attack of the NK cells.

Thus, the uses, methods and pharmaceutical compositions of the present invention are also suitable for the treatment of diseased cells which lack expression of HSP, e.g., HSP40, 60, 70, 90, and/or 110, on the cell surface and thus hitherto could not or not efficiently approached by immunotherapy. In a preferred embodiment, the cells to be treated are characterized by substantially lacking expression of HSP70 on the cell surface.

Under these circumstances, it is particularly preferred to induce or enhance the expression of HSP, especially HSP70 on the cell surface of the diseased cells or cells associated with a diseased tissue or organ in order to synergistically activate NK cells against those cells. Induction of the expression of HSP70 can be accomplished by hyperthermia, chemotherapy, radiotherapy or any combination thereof; see also the prior art referred to in context with HSP70, supra. Chemotherapy for tumors is varied, because there are so many different forms of this disease. Treatment may rely on a single anticancer medication—that is, single agent chemotherapy—or it may involve combination chemotherapy with a number of different anticancer drugs. Such drugs destroy cancer cells by preventing them from growing and dividing rapidly. In a preferred embodiment of the invention, said cytotoxic approaches induce apoptosis. An overview about apoptosis, the cell's intrinsic death program and key regulator of tissue homeostasis, is given in, e.g., Fulda and Debatin, Curr. Med. Chem. Anti-Canc. Agents 3 (2003), 253-262. International application WO03/086383 describes the use of a drug, in particular vincristine and paclitaxel, capable of inducing intracellular protein aggregation for the preparation of a pharmaceutical composition for the treatment of a tumor, a bacterial infection or a viral infection via induction of HSP70 expression on the cell surface of tumor/infected cells. Since these drugs are anti-tumor agents this embodiment provides the further advantage of a combination therapy which is supposed to exert synergistic effects.

Hence, the present invention also contemplates the use of the mentioned pharmaceutical compositions for sensitizing tumor cells for the activity of a cytotoxic second agent. This embodiment enables regimen to improve the effectiveness of chemotherapeutic agents which otherwise would be less effective or even not effective at all. Thus, the present invention also relates to the use of the above described pharmaceutical compositions for sensitizing tumor cells and infected cells for the activity of cytotoxic approaches such as apoptosis induction by chemotherapeutic drugs, γ-irradiation, and triggering of death receptors such as the CD95; see also supra. In particular, the present invention relates to pharmaceutical compositions for sensitizing tumor cells, in particular cells that are positively tested for the presence of a ligand for/of NKG2D, especially MICA/B and/or HSP, preferably HSP70 on their cell surface for the activity of chemotherapeutic agents. A similar approach has been described with respect to the presence and co-localization of a member of the anti-apoptotic Bcl-2-associated athanogene (Bag) family, especially Bag4 and HSP on the cell surface of diseased tissue or cells in international application WO2005/054868, the disclosure content of which is incorporated herein by reference, in particular with respect to the chemotherapeutic agents to be used. Another method for sensitizing antigen-presenting cells and membrane vesicles with immunogenic properties, and their use for vectoring and presenting antigens in vitro or in vivo and methods or compositions for treating cancers, infectious, parasitic or autoimmune diseases in particular are described in WO99/03499.

Other combination therapies may comprise, for example the use of cytokines, interleukins or preferably granzyme B. In particular, granzyme B has been shown to be most effective for the treatment of tumors, viral or bacterial infections or inflammatory diseases, wherein the tumor cells or the cells affected by said infection or inflammation express Hsp70 on their cell surface; see international application WO2004/018002 and appended Example 10.

The findings described herein have been further confirmed using a mouse model demonstrating that the heat shock protein HSP70 promotes mouse NK cell activity against tumors that express inducible NKG2D ligands; see Elsner et al., J. Immunol. 179 (2007) 5523-5533, which is incorporated herein by reference in its entirety.

Based on the findings described hereinbefore und illustrated in the appended Examples, the present invention also relates to a combination preparation comprising the ligand of NKG2D, a nucleic acid molecule encoding said ligand, or the inducer as defined hereinabove in combination with the NK activating agent and/or an agent capable of inducing expression of HSP70 on the cell surface. For example, in one embodiment the preparation may comprise a histone deacetylase inhibitor and an HSP70 peptide.

The present invention further provides methods of treating a subject having an undesirable condition associated with a disease as defined herein, comprising administering to the subject a therapeutically effective amount of any one of the pharmaceutical compositions and/or applying the medication plans described above. As mentioned, in principle any disease amenable to immunotherapy may be treated in accordance with the medical therapy of the present invention, such as infectious diseases, viral diseases, cancer to name the most prominent ones.

Infectious diseases that can be treated or prevented by the methods of the present invention are caused by infectious agents including, but not limited to, viruses, bacteria, fungi, protozoa and parasites. Bacterial diseases that can be treated or prevented by the methods of the present invention are caused by bacteria including, but not limited to, mycobacteria rickettsia, mycoplasma, neisseria and legionella. Protozoal diseases that can be treated or prevented by the methods of the present invention are caused by protozoa including, but not limited to, leishmania, kokzidioa, and trypanosoma. Parasitic diseases that can be treated or prevented by the methods of the present invention are caused by parasites including, but not limited to, chlamydia and rickettsia.

Viral diseases that can be treated or prevented by the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsachie virus, mumps virus, measles virus, rubella virus, polio virus, human immunodeficiency virus type I (HIV-I), and human immunodeficiency virus type II (HIV-II).

Cancers that can be treated or prevented by the methods of the present invention include, but are not limited to human sarcomas and carcinomas, e.g., fibrosarcoma, myxosarcoma, liposarcoma, chondrosarcoma, osteogenic sarcoma, carcinoma of the head/neck, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, glioblastoma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, and heavy chain disease.

In a specific embodiment the cancer is metastatic. In another specific embodiment, the subject having a cancer is immunosuppressed by reason of having undergone anti-cancer therapy (e.g., chemotherapy radiation) prior to administration of the compositions of the invention.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. A more complete understanding can be obtained by reference to the following specific examples. Several documents are cited throughout the text of this specification, sometimes by reference numbers in parenthesis. Full bibliographic citations may be found at the end of the specification immediately preceding the claims. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

### Examples

The examples which follow further illustrate the invention, but should not be construed to limit the scope of the invention in any way. Detailed descriptions of conventional methods, such as those employed herein can be found in the cited literature; see also “The Merck Manual of Diagnosis and Therapy” Seventeenth Ed. ed by Beers and Berkow (Merck & Co., Inc. 2003).

The practice of the present invention will employ, unless otherwise indicated, conventional techniques of cell biology, cell culture, molecular biology, transgenic biology, microbiology, recombinant DNA, and immunology, which are within the skill of the art. All references mentioned herein are incorporated in their entirety.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11, (2001), 98-107.

### Experimental Procedures

Animal Experiments

SCID (C.B-17/Ztm-scid) and SCID/beige (C.B-17/IcrHsd-scid-bg) mice were bred in the present colony under pathogen-free condition in individually ventilated cages. The SCID mice were originally obtained from Dr. H. J. Hedrich (Medizinische Hochschule Hannover, Germany) the SCID/beige mice were from Harlan Winkelmann (Borchen, Germany). Cages, bedding, and water were autoclaved, and a sterilized laboratory rodent diet was fed. All manipulations were done under aseptic conditions using a laminar flow hood. Female and male mice at an age between 12 and 20 weeks were used for experiments after excluding leaky mice by measuring serum immunoglobulins using an ELISA. All animal experiments had been approved by the local government and were in accordance with institutional guidelines for the welfare of animals. Tumor cells (1×10⁶ in 100 μl PBS) were injected subcutaneously into the flank of mice. Tumor growth was monitored every second day by palpation and size was recorded using linear calipers. Tumor volume was calculated by the formula V=πabc/2, where a, b, c are the orthogonal diameters. Animals were sacrificed before a tumor volume of 1 cm³ was reached, when a weight loss of more than 10% occurred, or when any behavioral signs of pain or suffering were observable. Autopsies of all animals were performed and the abdomen and thoracic cavity were examined systematically for the presence of metastases. The spleen of SCID mice was removed and placed in cell culture medium for subsequent immunological analyses. Parts of primary tumors were immediately frozen in liquid nitrogen for gene expression analyses. Tumor tissue and tissues suspicious for metastases were placed in phosphate-buffered 4% formalin for 16 h and then embedded in paraffin. Tissue sections (2.5 μm) were stained with hematoxylin and eosin for routine histological examinations to confirm the macroscopic observations. Immunohistochemical staining of the proliferation marker Ki67 was performed as described before (19). For flow cytometric analyses of tumor cells, tumor tissue was cut into small pieces and incubated in a 5 mg/ml collagenase solution (Sigma) at 37° C. for 90 min. Isolated cells were collected by centrifugation and resuspended in PBS before staining.

Gene Expression Analysis

RNA was prepared and Northern blots were hybridized with [³²P]dCTP-labeled probes as described (20). Autoradiograms were scanned for densitometry (Epson GT-8000 Scanner, ScanPack software, Biometra, Göttingen, Germany). Hybridization probes specific for MHC-linked rat Hsp70-1 (positions 2875 to 3070; accession no. X77207) (21) and human HSP70-2 genes (positions 2225 to 2407, accession no. M59830) (22) were derived from the 3′ untranslated region of the respective genes by genomic PCR amplification. A gene probe derived from the rhesus macaque MIC3 gene encompassing exons 2 to 5 (23) was used to detect MICA and MICB transcripts, which can be distinguished by size. Similarities of human and rhesus macaque MIC genes are about 90% and, therefore, probes are cross-reacting in Northern blot hybridizations. The human β-actin cDNA was purchased from Clontech (Heidelberg, Germany).

Target Cell Lines and Cell Culture

The human melanoma cell line Ge, the Ge-Hsp70 and Ge-con sublines derived therefrom (clones Ge-Hsp70-A, Ge-Hsp70-C, Ge-TCR-C, and Ge-GFP-B) (15), mouse fibroblast L cells, and transfected L cells were maintained in NaHCO₃-buffered DMEM supplemented with 10% FCS (Biochrom, Berlin, Germany), 2 mM L-glutamine, 1 mM sodium pyruvate, 50 μM 2-mercaptoethanol and antibiotics (100 U/ml penicillin, 100 μg/ml streptomycin) (Sigma) in Petri dishes for tissue culture (Sarstedt, Nümbrecht, Germany) at 37° C. in a 10% CO₂ atmosphere. Human erythroleukemia K562 and mouse lymphoma YAC-1 cells were maintained under the same conditions in Petri dishes for suspension cells (Sarstedt). To induce MICA and MICB expression, Ge cells were cultured in DMEM with 10 μM of the histone deacetylase inhibitor suberoylanilide hydroxyamic acid (SAHA) 20 h before the tests (24). L cells were co-transfected by electroporation with 30 μg of the cosmid A158 (25) containing the MIC3 gene of the rhesus macaque (Macaca mulatta) which is orthologous to human MICA, and with 1 μg of the dsRED vector (Clontech, Mountain View, USA), which confers resistance to geneticin (Invitrogen, Karlsruhe, Germany). After selection with geniticin (1 mg/ml) stable clones were established by limiting dilution, tested for presence of the cosmid by PCR using specific primers (5′-GCT TGC ATT CCC TCC AGG A-3′ (SEQ ID NO: 1) and 5′-TGG ACC CTC TGC AGC TGA TGT-3′ (SEQ ID NO: 2), length of specific PCR product 1396 bp). MICA mRNA expression was analyzed by Northern blot and cell surface expression by flow cytometry. Four independent clones were tested as target cells for human and rhesus lymphokine-activated killer (LAK) cells with similar results, confirming that MICA expression confers susceptibility to LAK cells. Clone K43 (L-MICA) was selected for the further studies.

Proliferation and Apoptosis of Ge Melanoma Cells

In vitro proliferation of Ge-Hsp70 and Ge-con cells was analysed by measurement of [methyl-3H]thymidine incorporation. 2×10⁴ cells per well in 200 μl DMEM were seeded in micro titer plates for cell culture (Sarstedt) in triplicates for each time point of measurement (12, 24, 48, and 72 h). 12 h before harvesting 1 μCi [methyl-3H]-thymidine (specific activity 5 Ci/mmol, Amersham) was added in 50 μl DMEM to the respective wells. Triplicates were harvested using a Titertek cell harvester 550 (Flow Laboratories, Irvine, UK). Incorporated radioactivity was determined by liquid scintillation counting using a Wallac MicroBeta Trilux counter. Apoptosis was induced in the melanoma cells by hypoxia and glucose starvation. To expose cells to hypoxic conditions Petri dishes were placed in a GasPak 100 system (Becton Dickinson) which normally serves for the culture of anaerobic bacteria. After 2 h the O₂ concentration in the system is less than 1% and the CO₂ concentration reaches 10% (Seip, W. F., and Evans, G. L. 1980. Atmospheric analysis and redox potentials of culture media in the GasPak System. J. Clin. Microbiol. 11 (1980), 226-233). The GasPak system was placed for 24 h in an incubator at 37° C. For glucose starvation the melanoma cell were cultured for 24 h in glucose-free DMEM (Sigma), which was supplemented as standard DMEM. Propidium iodide positive dead cells and apoptotic cells appearing in the sub G1 peak of DNA histograms were determined as described previously (20).

Preparation of Exosomes and Immunoblot Blot Analysis

The Ge-Hsp70 and Ge-con cell lines were grown to about 80% confluence before being cultured in fresh DMEM for 72 h. Viability was >95% as determined by trypan blue exclusion. The supernatant was harvested and exosomes were prepared as described (26) and analysed by SDS-PAGE. Immunoblotting was performed (26) using antibodies (Ab) specific for the inducible form of Hsp70 (clone C92F3A-5, mouse IgG1, SPA-810, StressGen, Biomol, Hamburg, Germany) and against Rab4 (sc-312, rabbit Ab, Santa Cruz, Biotechnology, Santa Cruz, USA).

Recombinant Proteins

A His-tagged recombinant HSP70 protein derived from the rat Hsp70-1 gene was described before (20). The rat Hsc70 gene was amplified by PCR (forward: 5′-GGATCCATGTCTAAGGGACCTGCAGTT-3′ (SEQ ID NO: 3) and reverse 5′-GAATTCGACTTAATCGACCTCTTCAATGGT-3′ (SEQ ID NO: 4)) from rat lymphocyte cDNA. The forward primer included a BamHI and the reverse primer an EcoRI restriction site at their 5′ ends. The amplification product was cloned into the pGEX-4T-2 expression vector (Amersham Biosciences, Braunschweig, Germany) via BamHI and EcoRI. A BamHI/SalI restriction fragment was isolated from this vector and recloned into the pQE30-1 expression vector (Qiagen, Hilden, Germany) in order to produce a His-tagged recombinant protein. The construct was sequenced to exclude missense mutations. The E. coli strain M15 (Qiagen) was transformed with this construct and used as host for overexpression of the His-tagged proteins. Induction and purification of the proteins was done as described previously (20). Recombinant human HSP70 (ESP-555, endotoxin concentration <50 EU/mg) was purchased from Stressgen.

Effector Cells and Effector Cell Culture

Splenocytes from SCID mice were obtained using a Tenbroeck homogeniser and erythrocytes were removed by incubation for 5 min in lysis buffer (155 mM NH₄Cl, 10 mM KHCO₃, 0.1 mM EDTA, pH 7.4-7.8). Afterwards the cells were used either directly as cytotoxic effector cells or cultured for 24 h in DMEM with 10% FCS and 20% supematant from concanavalin A stimulated lymphocytes before being used in ⁵¹Chromium release assays. Human effector cells were obtained from the peripheral blood of healthy voluntary laboratory co-workers by density gradient centrifugation on Biocoll separating solution (Biochrom). NK cells were isolated from peripheral blood mononuclear cells (PBMC) by magnetic cell sorting (MACS) using a negative selection kit (NK cell isolation Kit II, 130-091-152; Miltenyi Biotec, Bergisch-Gladbach, Germany). The kit contains a cocktail of antibodies against CD3, CD4, CD14, CD15, CD19, CD36, CD123, and CD235a. To obtain LAK cells the PBMC were cultured for 4 to 7 days in 5-ml Petri dishes for tissue culture (Sarstedt) at a density of 5 to 10×10⁶ cells/ml in DMEM supplemented with 100 U/ml IL-2 (Proleukin, Chiron, Amsterdam, Netherlands). NK cells enriched by MACS were cultured in 24-well plates for tissue culture (Sarstedt) at a density of 2×10⁶ cells/ml. To some cultures 2 μg/ml recombinant HSP70, recombinant HSC70, or the HSP70-derived peptide TKD (Bachem, Bubendorf, Switzerland) were added. TKD is good manufacturing practice (GMP) grade 14-mer peptide of the C-terminal substrate-binding domain of human HSP70 (TKDNNLLGRFELSG (SEQ ID NO: 5), aa 450-463) (27). Lipopolysaccharide (LPS) from E. coli was from Sigma (L4391) and added in a concentration of 10 ng/ml to some cultures.

⁵¹Chromium Release Assays

Target cells were labeled by incubating 1×10⁶ cells in 200 μl HEPES-buffered DMEM containing 100 μl FCS and 50 μCi Na₂⁵¹CrO₄ (ICN Biomedicals, Eschwege, Germany) for 1 h at 37° C. and washed three times with HEPES-buffered DMEM. Effector cells were added to 5×10^{3 51}Cr-labeled target cells in triplicate at ratios of about 100:1 to 1.5:1 for LAK cells and 10:1 to 0.6:1 for NK cells in 200 μl HEPES-buffered DMEM/10% FCS per well of round-bottomed micro titer plates. Spontaneous release was determined by incubation of target cells in the absence of effector cells. The micro titer plates were centrifuged for 5 min at 40×g and incubated at 37° C. for 4 h before being centrifuged again and supernatant and sediment were separately taken to determine radioactivity in each well using a Wallac MicroBeta Trilux counter (PerkinElmer Life Sciences, Köln, Germany). Percentage of specific lysis was calculated by subtracting percent spontaneous ⁵¹Cr release (20).

Flow Cytometry

Flow cytometry was done on a FACScan flow cytometer (Becton Dickinson, Heidelberg, Germany) using CellQuest software. Expression of intracellular HSP70, T cell receptor (TCR) β, and green fluorescent protein (GFP) in Ge-Hsp70 and Ge-con clones, respectively, was controlled regularly by flow cytometry as described before (15). Cell surface expression of HSP70 was examined on propidium iodide negative cells by a monoclonal antibody (mAb) (RPN 1197, mouse IgG1, multimmune, Regensburg, Germany) that has been reported to detect HSP70 on the plasma membrane (28). MICA/B cell surface expression was determined with the mAb BAMO1 reacting with human MICA and MICB (mouse IgG1, Immatics, Tübingen, Germany). For staining of human MHC class I molecules the mAb W6/32 (mouse IgG1, Serotec, Düsseldorf, Germany) was used. Intracellular granzyme B expression was analyzed with the mAb B18.1 (mouse IgG1, Alexis Biochemicals, Grünberg, Germany) after permeabilization of the cells with 0.25% saponin as described before for HSP70 (15). Secondary reagent for these unlabeled mouse IgG antibodies was polyclonal FITC-conjugated goat anti-mouse IgG (115-095-062; Jackson Laboratories, Dianova, Hamburg, Germany). Recombinant human and mouse NKG2D-Fc chimeric proteins (1299-NK, 139-NK) were purchased from R&D Systems (Wiesbaden, Germany) to detect cell surface expression of NKG2D ligands. Here polyclonal FITC-conjugated goat anti-human IgG (109-095-098; Jackson Laboratories, Dianova) was used as secondary reagent. The percentage of NK cells in the spleens of SCID mice was determined using the pan-NK cell marker DX5 (rat IgM, PE-conjugated, Caltag Laboratories, Hamburg, Germany). Human PBMC and NK cell enriched and depleted fractions were characterized by antibodies against CD3 (clone MEM 57, mouse IgG2a, FITC-conjugated, Immunotools, Friesoythe, Germany) CD4 (clone S3.5, mouse IgG2a, PE-conjugated, Caltag), CD8 (clone 3B5, mouse IgG2a, TC-conjugated, Caltag), CD14 (clone Tük4, mouse IgG2a, PE-conjugated, Caltag), CD16 (clone 3G3, mouse IgG1-TC-conjugated, Caltag), CD56 (clone MEM 188, mouse IgG2a, PE-conjugated, Caltag), CD94 (clone HP-3D9, mouse IgG1, FITC-conjugated, Becton Dickinson), NKG2D (clone 149810, mouse IgG1, PE-conjugated, R&D Systems). Isotype controls (mouse IgG1, IgG2a, and rat IgM) were purchased from Caltag.

Statistics

All data were analyzed using the software SAS version 9.1. Analysis of variance (ANOVA) for repeated measurements was carried out in all experimental designs. The different factors were incorporated into a two-way or three-way ANOVA involving interactions. Due to limited sample size in some experiments only a subgroup analysis (only the biologically most interesting factor levels) or a stratified analysis (by factor level) for a certain factor could be carried out. A significance level of α=0.05 was used.

Miscellaneous

Chemicals were from Sigma (Munich, Germany), Merck (Darmstadt, Germany) or Roth (Karlsruhe, Germany), if not indicated otherwise.

### Example 1

### Reduced Tumor Growth of HSP70 Overexpressing Melanoma Cells

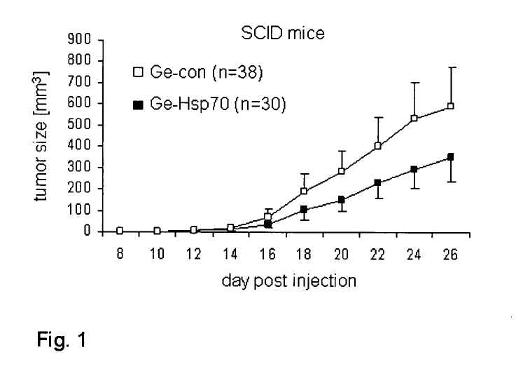

The human melanoma cell line Ge was transduced retrovirally to overexpress constitutively the normally stress-inducible MHC-linked rat Hsp70-1 (Hspa1) gene. The rat and human MHC-linked inducible HSP70 proteins are 96.3% identical and 98.4% similar, but they can be distinguished at the MRNA level by probes specific for the 3′ untranslated region. Control cell clones (Ge-con) were obtained by transduction with a rat TCRβ or GFP expression construct derived from the same vector. Both the Ge-Hsp70 and the Ge-con clones were previously described and characterized in detail by in vitro analyses (15). Ge-Hsp70 and Ge-con cells were injected subcutaneously into the flank of SCID mice, which lack T and B lymphocytes. Two clones of the Ge-Hsp70 cells (Ge-Hsp70-A and Ge-Hsp70-C) were used and two clones of the control cells (Ge-TCR-C and Ge-GFP-B) for these experiments. The primary tumors continued to grow progressively and at day 26 the first animals had to be sacrificed. Surprisingly, the growth of HSP70 overexpressing tumors was reduced compared to control tumors (Tab. 1). The tumor take after injection of Ge-Hsp70 cells at day 24 before the first animals had to be sacrificed was slightly decreased (73%) compared to Ge-con cells (86%). Most tumors grew locally but in several cases invasive growth of the primary tumors were observed and regional metastases in the mesenterium. Some metastases were additionally observed in the diaphragm and in regional lymph nodes. Intriguingly, the frequency of metastases (Tab. 1) was 21% for the Ge-con and 18% for the parental Ge tumors whereas none of the Ge-Hsp70 tumors gave rise to metastases. Thus, the permanent HSP70 overexpression appeared to reduce the malignancy of the Ge melanoma cells. Furthermore, even when tumors developed from Ge-Hsp70 cells, their growth rate was significantly reduced (p=0.0039, ANOVA) compared to tumors derived from Ge-con cells (FIG. 1).

### Example 2

### No Effect of Constitutive HSP70 Overexpression on Proliferation and Apoptosis

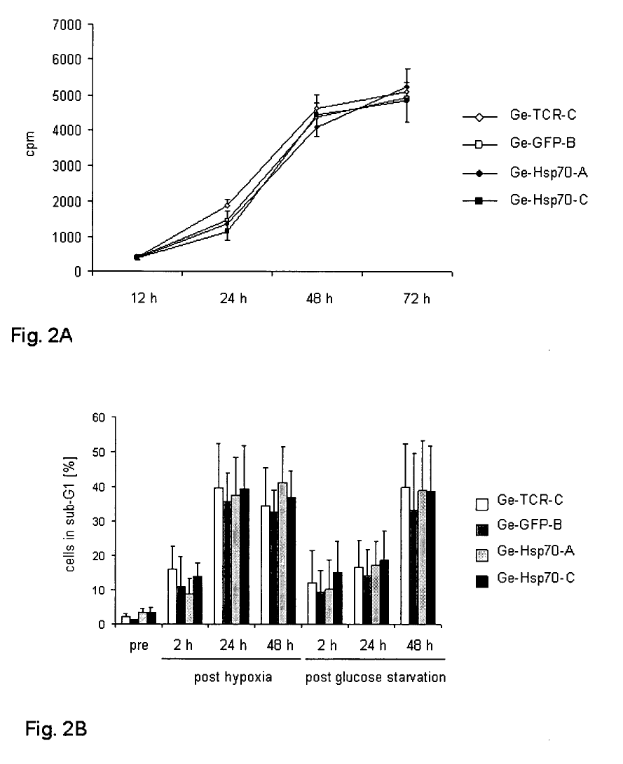

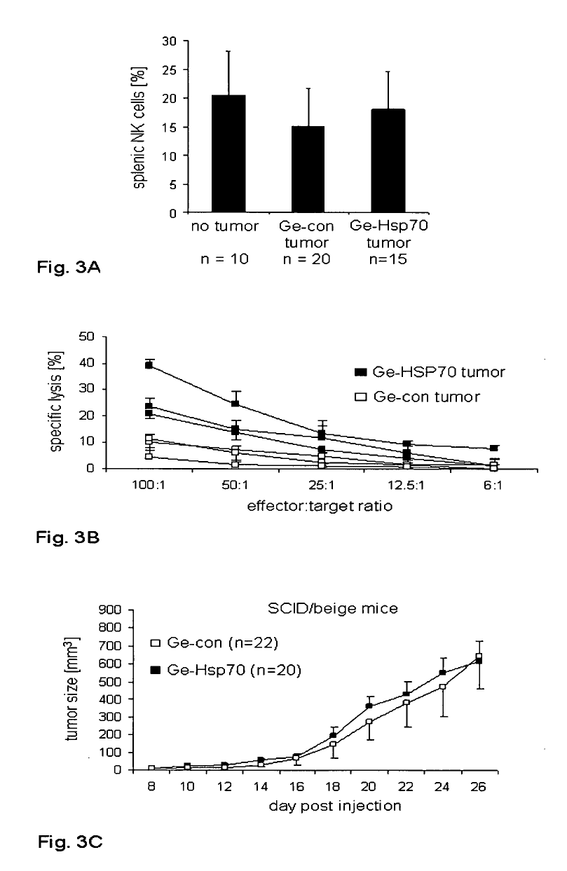

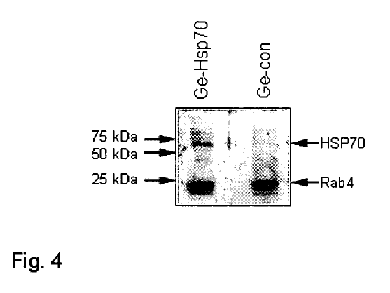

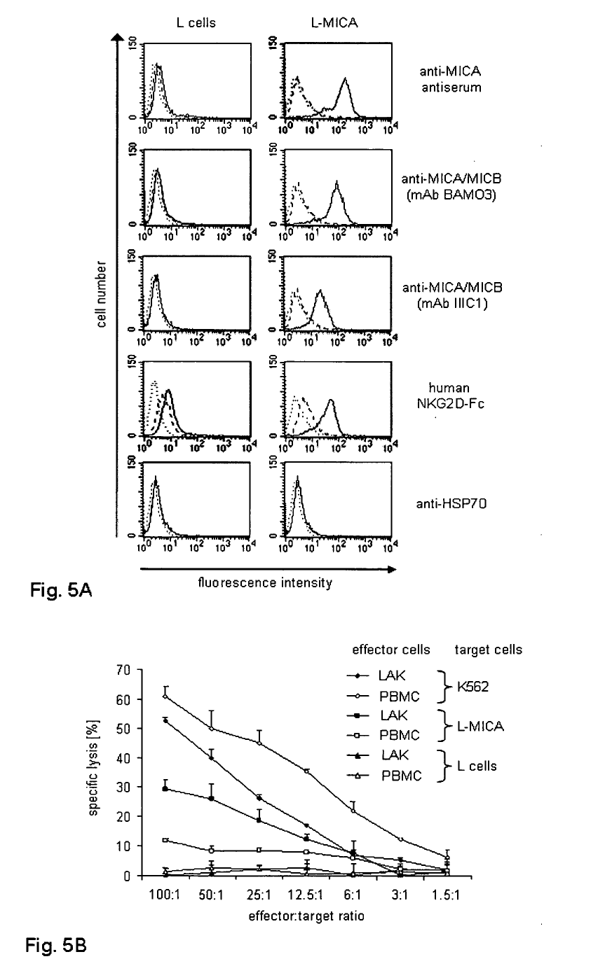

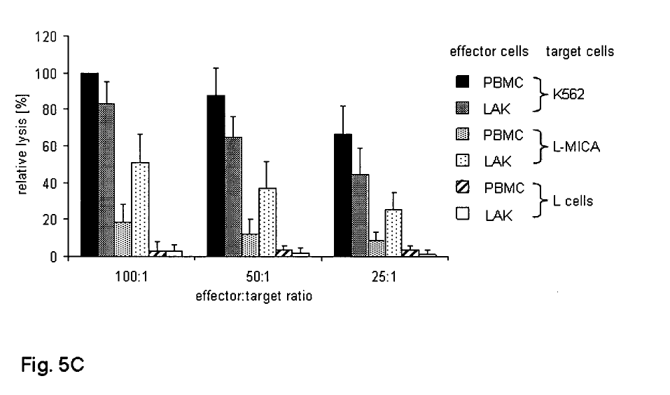

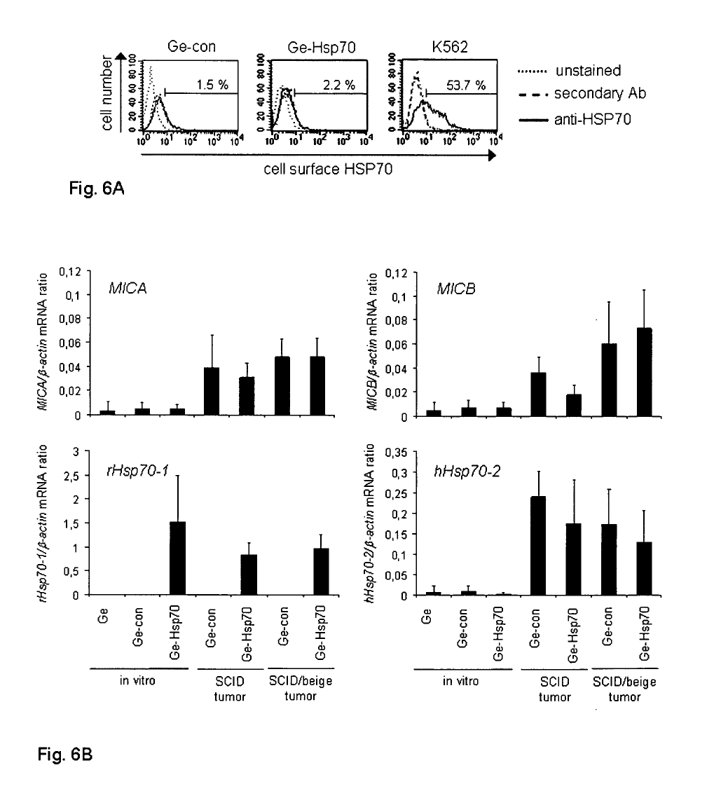

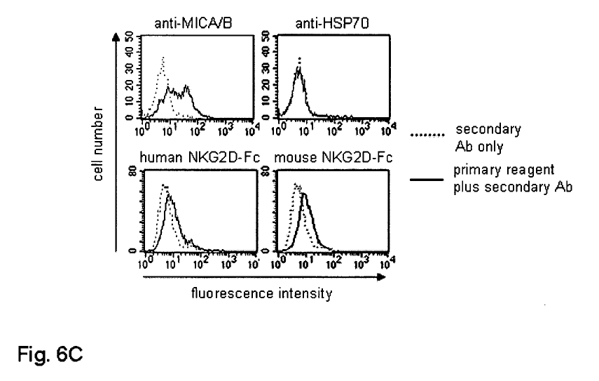

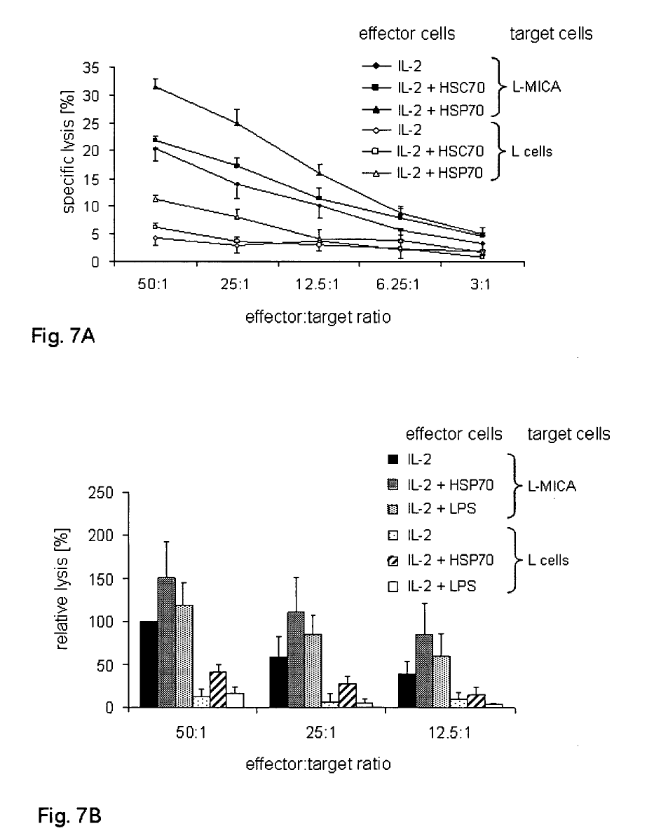

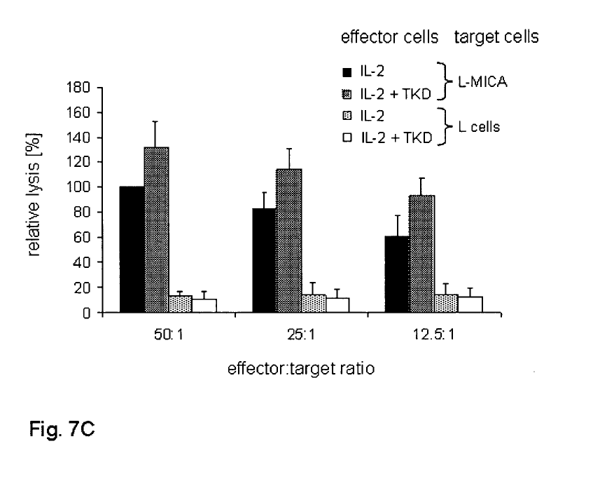

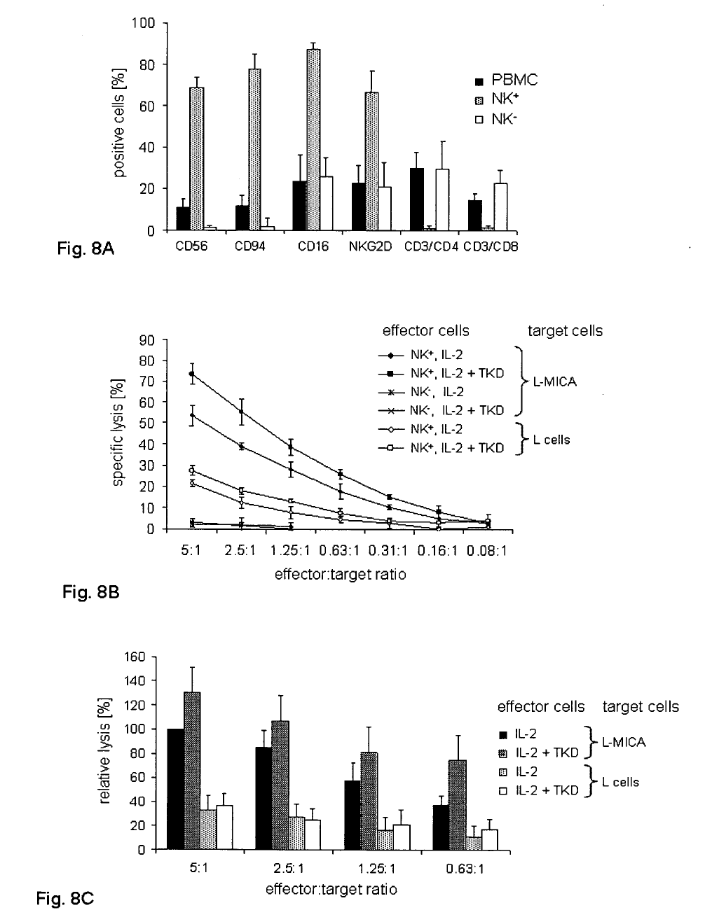

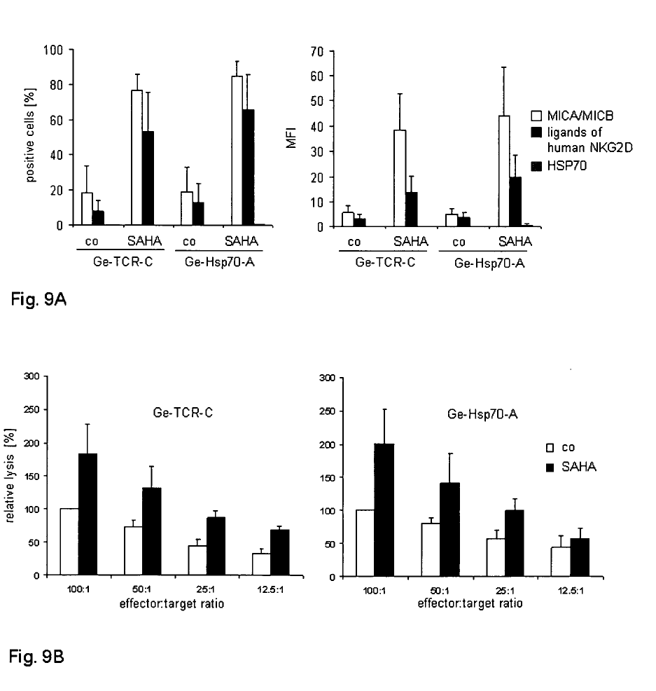

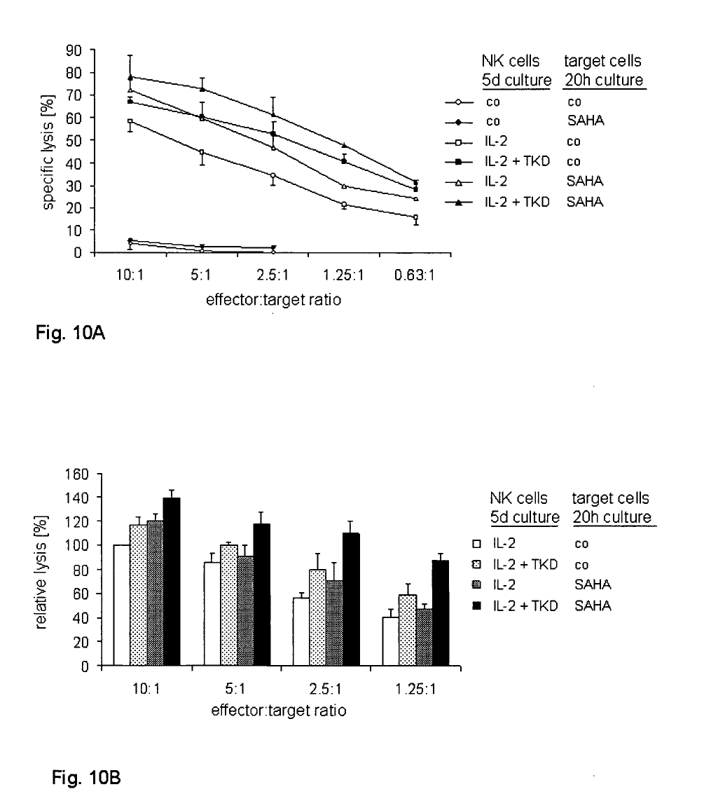

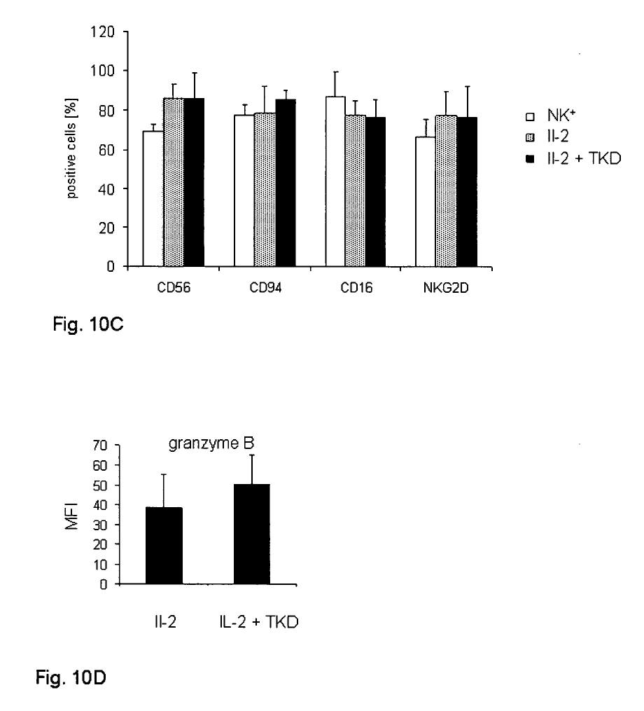

Histopathological evaluation and staining of tumors with the proliferation marker Ki67 did not reveal any major differences between Ge-Hsp70 and Ge-con-derived tumors. The proliferation in vitro was also not different between the HSP70 overexpressing and the control clones as determined by [3H]thymidine incorporation (FIG. 2A) and cell counting. Thus, the reduced growth of HSP70 overexpressing tumors could not be explained by differences in the proliferation rate. Therefore, cell death and apoptosis were analyzed after exposure of the cells to conditions that occur in tumors, such as hypoxia and glucose starvation. Again no difference was observed between the Ge-Hsp70 and the Ge-con cells when apoptosis was assessed by sub-G1 peak measurement (FIG. 2B) or cell death by propidium iodide staining after exposure to hypoxia or glucose-free medium for 24 h.

### Example 3

### Augmented NK Cell Activity in SCID Mice Bearing HSP70 Overexpressing Tumors

It was then hypothesized that the innate immune system, which is still present in SCID mice, might contribute to the partial control of the growth of HSP70 overexpressing melanomas. To address this question, the number and the cytotoxic activity of NK cells were analyzed from mice bearing HSP70 overexpressing or control tumors. The percentage of splenic NK cells of mice which rejected tumors and mice in which Ge-Hsp70 or Ge-con tumors grew did not differ markedly (FIG. 3A). Instead, the cytotoxic activity of splenocytes from mice with Hsp70 overexpressing tumors was augmented against the NK cell sensitive target cell line YAC-1 as exemplified in FIG. 3B. Although the cytotoxic activity of splenocytes derived from SCID mice against the Ge-Hsp70 and Ge-con cells was in vitro generally low, this result pointed towards a role of NK cells in controlling the growth of Ge-Hsp70 tumors.

### Example 4

### Tumor Growth of HSP70 Overexpressing Melanoma Cells is Not Reduced in SCID/Beige Mice

To further verify the role of NK cells in vivo the same cell clones as before were injected into SCID/beige mice, which lack in addition to T and B lymphocytes also functional NK cells. Now the tumor take (Tab. 2) and the growth of tumors in these mice (FIG. 3C) were similar after injection of Ge-Hsp70 and Ge-con cells. Tumors derived from Ge-Hsp70 cells lead to metastases in the same frequency as tumors from Ge-con cells (Tab. 2). These results clearly indicated that the differences observed in the SCID mice were due to the activity of NK cells, which partially controlled the growth of Ge-Hsp70-derived tumors and completely suppressed metastases.

### Example 5

### HSP70 Containing Exosomes are Released from HSP70 Overexpressing Melanoma Cells

Extracellular HSP70 has been shown to activate NK cells to specifically lyse HSP70 cell surface positive tumor cells (12). Therefore, it was determined whether HSP70 is released from the melanoma cells. It is known that cells, including tumor cells, can release exosomes which contain heat shock proteins (26, 29). Viable Ge-Hsp70 in contrast to Ge-con cells indeed released exosomes containing the inducible HSP70 (FIG. 4). In addition, HSP70 might be released in vivo also from necrotic areas which were present regularly in the tumors.

### Example 6

### No HSP70 Cell Surface but MICA/B Expression in Tumors

To determine whether HSP70 can also function as a target structure for NK cells (12), the expression of HSP70 on the melanoma cells was analyzed using an antibody suitable for HSP70 cell surface staining. The cultured Ge-Hsp70 as well as the Ge-con cells were negative for HSP70 cell surface staining (FIGS. 5A, 7B). Cells obtained from freshly prepared tumors from SCID or SCID/beige mice also did not demonstrate expression of HSP70 at the cell surface (FIG. 6A) although the transgenic rat Hsp70-1 mRNA was still found to be strongly expressed in the Ge-Hsp70-derived tumors (FIG. 6B). Therefore, it was asked whether other ligands for activating NK receptors might be present on the tumors. MICA and MICB molecules have been chosen. These human ligands among others had been shown to interact also with mouse activating NK receptor NKG2D (30-32). Expression of MICA and MICB mRNA was very low in vitro in Ge-Hsp70, Ge-con and parental Ge cells (FIG. 6B). This observation might explain the low cytotoxic activity of NK cells obtained from the SCID mice that was observed in vitro. In tumors, however, the expression of both MIC genes was clearly induced (FIG. 6B). Interestingly, MICA and MICB mRNA expression was higher in tumors grown in SCID/beige compared to those grown in SCID mice (MICA: p=0.0146 and MICB: p<0.0001). Differential expression of MICB mRNA in Ge-Hsp70 versus Ge-con tumors was modified by the host. It was decreased in SCID mice, whereas such a modification could not be found in SCID/beige mice (interaction: p=0.0437). A similar but statistically not significant effect seemed to be present also for MiCA mRNA expression. It is unlikely that these differences in MICA/B expression were due to different levels of cellular stress in the various tumors since the expression of the also stress-inducible endogenous human HSP70-2 mRNA did not vary similarly (FIG. 6B). In later experiments MICA/B molecules were confirmed to be expressed in vivo at the cell surface of tumor cells by staining single cell suspensions derived from tumors with anti-MICA/B mAb or recombinant human and mouse NKG2D (FIG. 6C).

These data suggested a functional role of MICA/B expression in the tumors. It was assumed that in SCID mice tumor cells, which express MICA/B, become targets for NK cells. Therefore, a selection against MICA and MICB expressing tumor cells might have occurred in SCID but not in NK cell deficient SCID/beige mice leading to reduced MICA and MICB mRNA expression levels in tumors in SCID mice. This selection pressure is apparently more important for HSP70 overexpressing tumors than for control tumors, because HSP70 appears to activate NK cells against tumors. Together these findings indicated that HSP70 released from HSP70 overexpressing tumor cells, could activate NK cells to kill MICA/B expressing tumor cells leading to reduced tumor growth and suppression of metastases.

### Example 7

### HSP70 and the HSP70-Derived Peptide TKD Activate Human PBMC to Kill MICA Expressing Target Cells in Vitro

Next the hypothesis was tested deduced from the animal experiments with human effector cells instead of mouse cells. Therefore, a series of in vitro experiments were performed. From previous experiments mouse L cells were available transfected with a cosmid containing the MICA gene derived from the rhesus macaque (Macaca mulatta). These L-MICA cells express MICA at the cell surface (FIG. 5A). However, they do not express HSP70 at the plasma membrane (FIG. 5A). L-MICA cells can be killed by human LAK cells, but hardly by unstimulated PBMC (FIGS. 5B, 5C). Similar results were obtained with further clones transfected with the cosmid containing the MICA gene or a MICA cDNA expression construct. Control cells transfected with vectors only did not differ from parental L cells in these experiments. The MICA expressing L cells were used as targets for HSP70 activated killer cells in further experiments.

Human PBMC were cultured for seven days in the presence of low dose IL-2 (100 U/ml) and added to parallel cultures recombinant HSP70 molecules, either 2 μg/ml of the stress-inducible HSP70 or the constitutively expressed HSC70. The IL-2 treated PBMC lysed the MICA expressing L cells, but hardly the control L cells (FIG. 7A). HSP70 in contrast to HSC70 treatment provided an additional stimulatory effect leading to further increased lysis of the L-MICA cells by IL-2 treated PBMC (FIG. 7A). Thus, HSP70 but not HSC70 was able to further activate PBMC to kill MICA expressing target cells. Both proteins, HSP70 and HSC70, were prepared as recombinant proteins in E. coli and can be expected to be contaminated with LPS. Therefore, additionally recombinant “low endotoxin” (<50 EU/mg) human HSP70 were used and similar effects were observed. Furthermore, in parallel the effects of HSP70 and LPS (10 ng/ml) on LAK cell stimulation were tested. In these tests HSP70 treatment significantly increased (p=0.0374) the capacity of LAK cells to kill L-MICA cells (FIG. 7B). To avoid LPS contamination of HSP70 preparations completely, the HSP70-derived peptide TKD (TKDNNLLGRFELSG) was used, which is produced by chemical synthesis under GMP conditions. The peptide TKD was shown before to be equivalent to the full length HSP70 in its ability to stimulate NK cells to lyse HSP70 cell surface positive target cells (27). It was found that PBMC, which were stimulated for 7 days with IL-2 and TKD (2 μg/ml), lysed L-MICA cells significantly better (p=0.0012) than cells cultured with IL-2 only confirming the data obtained with recombinant full-length HSP70.

### Example 8

### NK Cells are the Effector Cells Which are Activated by the HSP70 Peptide TKD to Kill MICA Expressing Target Cells in Vitro

Based on the in vivo data shown above, it was assumed that NK cells are the cytotoxic effector cells among the PBMC which are activated by IL-2 plus HSP70 or IL-2 plus TKD. However, other cells such as CD8⁺ T cells or γδT cells can express the MICA and MICB receptor NKG2D and might contribute to the effects observed in vitro. To test this hypothesis NK cells were isolated from the peripheral blood of voluntary donors by MACS before the in vitro culture (FIG. 8A). In parallel, the cytotoxic activity of the NK cell depleted cell population was tested. The NK cell enriched fraction killed L-MICA cells much better than L cells (FIG. 8B) and TKD stimulation was able to significantly improve (p<0.0001) the activity of the NK cells against L-MICA cells (FIG. 8C). Stimulation of NK cell depleted PBMC by IL-2 or IL-2 plus TKD did not result in a cell population that was able to kill L-MICA cells (FIG. 8B). Even at high effector:target ratios (up to 200:1) the lysis of L-MICA as well as L cells remained very low. Thus, NK cells were the necessary cytotoxic cells which execute the TKD effect on MICA expressing target cells.

### Example 9

### MICA/B Expression on Ge Melanoma Cells and HSP70 Peptide TKD Activation of NK Cells Lead Synergistically to High Killing of Tumor Cells

Next it was investigated whether MICA/B expressing human Ge melanoma cells, which were used for the in vivo experiments, can become a target for HSP70-activated human NK cells. It was shown before that these cells do express MHC class I molecules (15, 16). However, it is known that the expression of MICA or MICB as ligands for the activating NK cell receptor NKG2D can overcome the MHC class I mediated inhibition of NK cells (18, 33). MICA/B were induced on Ge-con as well as on Ge-Hsp70 cells by histone deacetylase inhibitors (24) such as trichostatin A or suberoylanilide hydroxyamic acid (SAHA) (FIG. 9A). Importantly, a HSP70 cell surface expression was not found after this treatment. The treated cells were significantly more susceptible to lysis by LAK cells than untreated cells (FIG. 9B). Isolated NK cells were cultured for 5 days without IL-2, with IL-2, or with IL-2 plus TKD before they were used as effector cells for Ge-con target cells that were cultured before under standard conditions or for 20 h in the presence of a histone deacetylase inhibitor (10 μM SAHA). As demonstrated in FIG. 10A, IL-2 was indispensable to obtain NK cells, which were able to kill Ge-con melanoma cells. TKD treatment of NK cells increased their capability to lyse target cells (p=0.0055; for all results in this paragraph three-way ANOVA was used for effector:target ratios 2.5:1 and 1.25:1). SAHA treatment of Ge target cells augmented their susceptibility to NK cells (p<0.0001). The combination of TKD treatment of NK cells and SAHA treatment of target cells acted synergistically (p value for interaction p=0.0003) and resulted in an increased killing of target cells in these experiments especially at lower effector:target ratios (FIG. 10B). Similar results were obtained after stimulation of NK cells with full-length recombinant HSP70 and for Ge-Hsp70 cells as target cells. Thus, in accord with the mouse in vivo experiments HSP70 and MICA/B can jointly augment in vitro killing of tumor cells by human NK cells.

### Example 10

### HSP70 Peptide TKD Stimulation of NK Cells Increases Granzyme B Expression

To further analyze the effect of the HSP70 peptide TKD on NK cells the expression of the NK cell markers CD56, CD94, CD16 and of NKG2D on freshly isolated NK cells was determined and after 5 d culture in the presence of IL-2 or IL-2 plus TKD (FIG. 10C). After the cultures the percentage of CD56 positive cells was increased. However, no difference was observed between IL-2 and IL-2 plus TKD treatment. The mean fluorescence intensity of both NKG2D and CD94 increased after IL-2 and TKD treatment, but no significant difference was detectable compared to the IL-2 stimulation alone. Thus, with these markers a major change of the NK cell phenotype during the cultures with TKD was not observed. Similar results were obtained after stimulation of NK cells with full-length recombinant HSP70. It was then speculated that TKD treatment might augment the expression of cytotoxic effector molecules in the activated NK cells. Indeed, a flow cytometric analysis of intracellular granzyme B indicated a tendency towards increased expression due to IL-2 plus TKD stimulation compared to IL-2 stimulation alone (FIG. 10D). Thus, the HSP70 effects on NK cells that was observed might be explainable at least in part by an increased expression of cytotoxic effector molecules.

Discussion

Components of innate and adaptive immunity evolved in the context of the evolutionary much older stress response system. The stress response is destined to maintain survival of cells that have been exposed to adverse environmental conditions. For being successful the immune system needs to be able to destroy target cells, even during an ongoing stress response initiated for cellular protection. Moreover, stressed cells, e. g. virus-infected cells or tumor cells, appear to be usually a more appropriate target for cytotoxic effector cells of the immune system than unstressed cells. It was shown before that HSP70, which is known to protect cells efficiently against various adverse conditions, falls to protect in vitro against specific cytotoxic effector mechanisms of CTL mediated in the granule-exocytosis pathway (15, 16, 20). Thus, cytotoxic effector mechanisms of the cellular immune system seem to dominate over the protective stress response. In accord with this assumption it has been suggested that certain components of the stress response system function as “danger signals” triggering the initiation of an immune response (13, 14). Typical exogenous danger signals are the pathogen-associated molecular patterns (PAMPs) which are recognized by PAMP receptors on cells of the innate immune system. Endogenous danger signals are produced by the organism that is exposed to danger. They can appear at the cell surface or become released. Some elements of the cellular stress response, such as stress-inducible HSP70, indeed appear to be able to activate and to connect both, innate and adaptive immune reactions (10, 11) and to fulfill the criteria of endogenous danger signals. Further examples of potential endogenous danger signals are the ligands of the NKG2D receptor. NKG2D has been shown to serve as an activating receptor triggering NK cell responses against tumors (18) and expression of NKG2D ligands in tumors was reported to induce tumor rejection (32, 34, 35). NKG2D ligands include in humans MICA and MICB. These and further NKG2D ligands appear to be up-regulated in response to cellular (17) or genotoxic stress (36) and signal the immune system the presence of potentially dangerous cells (37).

The experiments performed in accordance with the present invention show that two stress-inducible endogenous danger signals, HSP70 and MICA/B, synergistically improve the cytotoxic activity of NK cells against tumor cells. Human PBMC cultured in presence of HSP70 acquired an increased cytotoxic activity against tumor cells which were either transfected to express the NKG2D ligand MICA or in which the endogenous MICA/B was induced by pharmacological means. The latter experiments showed that a combination of both treatments acted synergistically and resulted in a significantly enhanced killing of target cells. Cell separation experiments clearly demonstrated that NK cells are required to execute the cytotoxic effect that was stimulated by HSP70. HSP70 might either act directly on NK cells (38) or on other cells. Dendritic cells, e. g., are known to express heat shock protein receptors (10, 11) and to cross-talk to NK cells (39).

The stimulatory effect of HSP70 on NK cells appears to be a specific property of the stress-inducible HSP70 in contrast to the constitutively expressed HSC70, as shown by the direct comparison of recombinant HSP70 and HSC70 in stimulation assays. In accord with this observation, the peptide TKD, which is derived from HSP70 and is not present in the HSC70 protein (27), was able to substitute for the full-length HSP70 protein.

Effects of recombinant proteins on cells of the immune system are always suspicious to be caused by LPS contamination. However, HSC70 which was produced under the same conditions as HSP70 had not the same effect on the NK cells. Furthermore, commercially available “low endotoxin” HSP70 was still able to stimulate the NK cells. Even more importantly, the HSP70 peptide TKD which was produced by chemical synthesis under GMP conditions also stimulated NK cells ruling out that only LPS effects were observed.

At this point it remains to be elucidated how HSP70 or the peptide TKD activates NK cells. A significant increase of the NKG2D positive cell population was not observed during the culture of NK cells with IL-2 plus TKD compared to IL-2 only. The expression level of NKG2D was also not augmented significantly as analyzed by flow cytometry. NKG2D has been suggested to serve primarily as a receptor for NK cell granule-mediated cytotoxicity (18). Consistently, a tendency towards higher expression levels of the cytotoxic effector protease granzyme B in NK cells exposed to TKD was noted. HSP70 and TKD might increase the cytotoxic activity of NK cells by inducing the expression of cytotoxic effector molecules. However, it is not clear whether this is the only or the main mechanism by which HSP70 can increase the activity of NK cells.

Nonetheless, the activation of NK cells by HSP70 against MICA/B-expressing target cells appears to be relevant also in vivo for tumor immune surveillance. The growth of HSP70 overexpressing Ge melanoma cells was significantly reduced in SCID mice. Moreover, these tumors in contrast to control tumors did not grow invasively and did not give rise to regional metastases. These effects could clearly be attributed to NK cells in SCID mice, since tumor growth and rate of metastases of HSP70 overexpressing tumors was not different from control tumors in SCID/beige mice, which lack in addition to B and T lymphocytes also functional NK cells.

It was shown before that the HSP70 overexpressing Ge melanoma cells compensatory downregulate HSC70 expression (15). Therefore, the HSC70 expression is basically shifted towards the expression of HSP70 in these cells (15).

In accordance with the present experiments it could be shown now that HSP70 containing exosomes are released from HSP70 overexpressing melanoma cells. The increased amount of released HSP70 could subsequently result in a better activation of NK cells in mice bearing HSP70 overexpressing tumors, since NK cells appear to be activated by HSP70 but not by HSC70. It is important to notice that all the HSP70 present in tumors was of eukaryotic source. Therefore, it can be excluded that LPS effects could be relevant for the observations made in the animal model.

The Ge melanoma cells express MICA and MICB in vivo, but hardly in vitro. This expression in tumors appears to be functionally relevant in SCID mice, since the expression level of both genes, MICA and MICB, was reduced in HSP70 overexpressing tumors grown in SCID compared to SCID/beige mice. This reduced MICA/B expression is interpreted as an example of “cancer immunoediting”, as suggested by Schreiber and colleagues (40). MICA/B expressing tumor cells become a preferential target for HSP70 activated NK cells present in SCID mice leading to a loss of those cells. This NK cell activity explains the reduced size of HSP70-overexpressing tumors. Although the reduced expression of MICA/B points also towards a potential immune escape mechanism, the HSP70 overexpression in primary tumors and subsequent activation of NK cells, which can kill MICA/B expressing tumor cells, was obviously sufficient to completely suppress invasive growth and regional metastases.

It might be important that the expression of NKD2D ligands MICA/B was endogenously regulated in the tumors in the present model. Strong ectopic expression of MICA/B was reported to result in an over-stimulation of NKG2D expressing cells and in a consecutive down-regulation of NKG2D and inactivity of NK cells and CD8⁺ T cells (41-43).

Numerous studies used tumor-derived heat shock proteins, including glucose regulated protein (GRP) 94 (gp96, HSP96) and HSP70, for immunotherapy (10). Although the focus was on the induction of CTL responses, it has been noticed that also NK cell depletion can abrogate the efficacy of immunization with gp96 or HSP70 (44). Furthermore, a perforin-dependent NK cell activity was reported to be required to induce a CTL-mediated rejection of tumor cells which were engineered to secrete gp96 (45). NK cells also seem to be necessary for an adjuvant-like activity of HSP70 in the induction of CTL responses (46). Consistent with these reports it was recently observed that patients treated with autologous tumor-derived HSP96 undergo a significant boost of NK cell activity (47). Thus, an initial NK cell-mediated lysis of tumor cells and/or NK-released cytokines might be required for efficient priming of tumor-specific CTL. Interestingly, it was described before that HSP70 (48) and the HSP70-derived peptide TKD (27) can stimulate NK cells to specifically lyse tumor cells which express HSP70 at the plasma membrane (12). HSP70 serves in this case as stimulatory molecule and as target structure for NK cells although it is not clear how HSP70 is expressed in the plasma membrane. The present results describing the activation of NK cells by HSP70 and TKD are in agreement with these data. However, the tumor cells analyzed here did neither in vitro nor in vivo express HSP70 at the cell surface. Instead it is shown that transmembrane proteins MICA/B, which are ligands for the well-defined activating NK receptor NKG2D, can serve as target structure for HSP70 and TKD-stimulated NK cells.

The adoptive transfer of TKD-activated NK cells is a promising new immunotherapy for tumors that express HSP70 at the plasma membrane, which has been successfully evaluated in preclinical animal models (49) and also in a phase-I-clinical trial (50). Since not all tumors express HSP70 at the cell surface, the finding of the present invention that HSP70 stimulated NK cells can use NKG2D ligands as target structures substantially increases the spectrum of patients who might profit from this kind of immunotherapy. It has to be evaluated whether a combination of HSP70 and NKG2D ligand expression on tumor cells improves the result of the therapy. A combination of hyperthermia to induce HSP70 and pharmacotherapy with histone deacetylase inhibitors to induce MICA/B (24) might also be a promising strategy to improve NK cell activity against tumors. Importantly, NK cells appear not only to function as cytotoxic effector cells but also to contribute essentially to the induction of a subsequent CTL response (39, 45, 46). Therefore, it is even more essential to understand which combination of signals from tumor cells results in the best activation of NK cells. The present results of enhanced NK cell activity in the presence of two danger signals might help to improve strategies for cancer immunotherapy. Additionally, they provide further insight into the various roles of HSP70 and MICA/B in tumor biology.

### REFERENCES

1. Beere H M. Death versus survival: functional interaction between the apoptotic and stress-inducible heat shock protein pathways. J Clin Invest 2005;115:2633-9.

2. Calderwood S K, Khaleque M A, Sawyer D B, Ciocca D R. Heat shock proteins in cancer: chaperones of tumorigenesis. Trends Biochem Sci 2006;31:164-72.

3. Nylandsted J, Rohde M, Brand K, Bastholm L, Elling F, Jäättelä M. Selective depletion of heat shock protein 70 (Hsp7O) activates a tumor-specific death program that is independent of caspases and bypasses Bcl-2. Proc Natl Acad Sci USA 2000;97:7871-6.

4. Gurbuxani S, Bruey J M, Fromentin A, et al. Selective depletion of inducible HSP70 enhances immunogenicity of rat colon cancer cells. Oncogene 2001;20:7478-85.

5. Nylandsted J, Wick W, Hirt U A, et al. Eradication of glioblastoma, and breast and colon carcinoma xenografts by Hsp70 depletion. Cancer Res 2002;62:7139-42.

6. Menoret A, Patry Y, Burg C, Le Pendu J. Co-segregation of tumor immunogenicity with expression of inducible but not constitutive hsp70 in rat colon carcinomas. J Immunol 1995;155:740-7.

7. Melcher A, Todryk S, Hardwick N, Ford M, Jacobson M, Vile R G Tumor immunogenicity is determined by the mechanism of cell death via induction of heat shock protein expression. Nat Med 1998;4:581-7.

8. Clark P R, Menoret A. The inducible Hsp70 as a marker of tumor immunogenicity. Cell Stress Chaperon 2001;6:121-5.

9. Massa C, Guiducci C, Arioli I, Parenza M, Colombo M P, Melani C. Enhanced efficacy of tumor cell vaccines transfected with secretable hsp70. Cancer Res 2004;64: 1502-8.

10. Srivastava P. Roles of heat-shock proteins in innate and adaptive immunity. Nat Rev Immunol 2002;2: 185-94.

11. Nicchitta C V. Re-evaluating the role of heat-shock protein-peptide interactions in tumour immunity. Nat Rev Immunol 2003;3:427-32.

12. Multhoff G, Botzler C, Jennen L, Schmidt J, Ellwart J, Issels R. Heat shock protein 72 on tumor cells: a recognition structure for natural killer cells. J Immunol 1997;158:4341-50.

13. Matzinger P. Tolerance, danger, and the extended family. Annu Rev Immunol 1994;12:991-1045.

14. Todryk S M, Melcher A A, Dalgleish A G, Vile R G. Heat shock proteins refine the danger theory. Immunology 2000;99:334-7.

15. Dressel R, Grzeszik C, Kreiss M., et al. Differential effect of acute and permanent heat shock protein 70 overexpression in tumor cells on lysability by cytotoxic T lymphocytes. Cancer Res 2003;63:8212-20.

16. Dressel R, Lübbers M, Walter L, Herr W, Günther E. Enhanced susceptibility to cytotoxic T lymphocytes without increase of MHC class I antigen expression after conditional overexpression of heat shock protein 70 in target cells. Eur J Immunol 1999;29:3925-35.

17. Groh V, Bahram S, Bauer S, Herman A, Beauchamp M, Spies T. Cell stress-regulated human major histocompatibility complex class I gene expressed in gastrointestinal epithelium. Proc Natl Acad Sci USA 1996;93:12445-50.

18. Hayakawa Y, Smyth M J. NKG2D and cytotoxic effector function in tumor immune surveillance. Semin Immunol 2006;18:176-85.

19. Alves F, Contag S, Missbach M, et al. An orthotopic model of ductal adenocarcinoma of the pancreas in severe combined immunodeficient mice representing all steps of the metastatic cascade. Pancreas 2001;23:227-35.

20. Dressel R, Elsner L, Quentin T, Walter L, Günther E. Heat shock protein 70 is able to prevent heat shock-induced resistance of target cells to CTL. J Immunol 2000;164: 2362-71.

21. Walter L, Rauh F, Günther E. Comparative analysis of the three major histocompatibility complex-linked heat shock protein 70 (Hsp7O) genes of the rat. Immunogenetics 1994;40:325-30.

22. Milner C M, Campbell R D. Structure and expression of the three MHC-linked HSP70 genes. Immunogenetics 1990;32:242-51.

23. Seo J W, Bontrop R, Walter L, Günther E. Major histocompatibility complex-linked MIC genes in rhesus macaques and other primates. Immunogenetics 1999;50:358-62.

24. Skov S, Pedersen M T, Andresen L, Straten P T, Woetmann A, Odum N. Cancer cells become susceptible to natural killer cell killing after exposure to histone deacetylase inhibitors due to glycogen synthase kinase-3-dependent expression of MHC class I-related chain A and B. Cancer Res 2005;65:11136-45.

25. Seo J W, Walter L, Günther E. Genomic analysis of MIC genes in rhesus macaques. Tissue Antigens 2001;58:159-65.

26. Gastpar R, Gehrmann M, Bausero M A, et al. Heat shock protein 70 surface-positive tumor exosomes stimulate migratory and cytolytic activity of natural killer cells. Cancer Res 2005;65:5238A47.

27. Multhoff G, Pfister K, Gehrmann M, et al. A 14-mer Hsp70 peptide stimulates natural killer (NK) cell activity. Cell Stress Chaperon 2001 ;6:337-44.

28. Botzler C, Li G, Issels R D, Multhoff G. Definition of extracellular localized epitopes of Hsp70 involved in an NK immune response. Cell Stress Chaperon 1998;3:6-11.

29. Thery C, Regnault A, Garin J, et al. Molecular characterization of dendritic cell-derived exosomes. Selective accumulation of the heat shock protein hsc73. J Cell Biol 1999;147:599-610.

30. Diefenbach A, Jamieson A M, Liu S D, Shastri N, Raulet D H. Ligands for the murine NKG2D receptor: expression by tumor cells and activation of NK cells and macrophages. Nat Immunol 2000;1:119-26.

31. Wolan D W, Teyton L, Rudolph M G, et al. Crystal structure of the murine NK cell-activating receptor NKG2D at 1.95 Å. Nat Immunol 2001;2: 248-54.

32. Friese M A, Platten M, Lutz S Z, et al. MICA/NKG2D-mediated immunogene therapy of experimental gliomas. Cancer Res 2003;63:8996-9006.

33. Bauer S, Groh V, Wu J, et al. Activation of NK cells and T cells by NKG2D, a receptor for stress-inducible MICA. Science 1999;285:727-9.

34. Cerwenka A, Bakker A B, McClanahan T, et al. Retinoic acid early inducible genes define a ligand family for the activating NKG2D receptor in mice. Immunity 2000;12:721-7.

35. Diefenbach A, Jensen E R, Jamieson A M, Raulet D H Rael and H60 ligands of the NKG2D receptor stimulate tumour immunity. Nature 2001 ;413:165-71.

36. Gasser S, Orsulic S, Brown E J, Raulet D H. The DNA damage pathway regulates innate immune system ligands of the NKG2D receptor. Nature 2005;436:1186-190.

37. Gasser S, Raulet D H. The DNA damage response arouses the immune system. Cancer Res 2006;66:3959-62.

38. Gross C, Hansch D, Gastpar R, Multhoff G. Interaction of heat shock protein 70 peptide with NK cells involves the NK receptor CD94. Biol Chem 2003;384:267-79.

39. Degli-Esposti M A, Smyth, M J. Close encounters of different kinds: dendritic cells and NK cells take centre stage. Nat Rev Immunol 2005;5:112-24.

40. Dunn G P, Old L J, Schreiber R D. The three Es of cancer immunoediting. Annu Rev Imnunol 2004;22:329-60.

41. Wiemann K, Mittrücker H W, Feger U, et al. Systemic NKG2D down-regulation impairs NK and CD8 T cell responses in vivo. J Immunol 2005;175:720-9.

42. Oppenheim D E, Roberts S J, Clarke S L, et al. Sustained localized expression of ligand for the activating NKG2D receptor impairs natural cytotoxicity in vivo and reduces tumor immunosurveillance. Nat Immunol 2005;6:928-37.

43. Coudert J D, Zimmer J, Tomasello E, et al. Altered NKG2D function in NK cells induced by chronic exposure to NKG2D ligand-expressing tumor cells. Blood 2005;106:1711-7.

44. Tamura Y, Peng P, Liu K, Daou M, Srivastava P K. Immunotherapy of tumors with autologous tumor-derived heat shock protein preparations. Science 1997;278: 117-20.

45. Strbo N, Oizumi S, Sotosek-Tokmadzic V, Podack E R. Perforin is required for innate and adaptive immunity induced by heat shock protein gp96. Immunity 2003;18:381-90.

46. Massa C, Melani C, Colombo M P. Chaperon and adjuvant activity of hsp70: different natural killer requirement for cross-priming of chaperoned and bystander antigens. Cancer Res 2005;65:7942-9.

47. Pilla L, Squarcina P, Coppa J, et al. Natural killer and NK-Like T-cell activation in colorectal carcinoma patients treated with autologous tumor-derived heat shock protein 96. Cancer Res 2005;65:3942-9.

48. Multhoff G, Mizzen L, Winchester C C, et al. Heat shock protein 70 (Hsp70) stimulates proliferation and cytolytic activity of natural killer cells. Exp Hematol 1999;27:1627-36.

49. Stangl S, Wortmann A, Guertler U, Multhoff G. Control of metastasized pancreatic carcinomas in SCID/beige mice with human IL-YrKD-activated NK cells. J Immunol 2006;1 76:6270-6.

50. Krause S W, Gastpar R, Andreesen R, et al. Treatment of colon and lung cancer patients with ex vivo heat shock protein 70-peptide-activated, autologous natural killer cells: a clinical phase I trial. Clin Cancer Res 2004;10:3699-707.

1. A method for treating a disease in a subject which comprises administering to the subject, wherein said disease involves cells which express or are induced to express a ligand for NK cell receptor NKG2D on the cells' surface.

2. The method of claim 1, wherein said disease is a tumor or infectious disease.

3. The method of claim 1, wherein said ligand is MHC class I chain-related (MIC) A or B.

4. The method of claim 1, wherein said NK cells are activated prior to administration to the subject or are administered in conjunction with an activator of NK cells.

5. The method of claim 1, wherein said activator comprises HSP70 or a peptide derived therefrom.

6. The method of claim 5, wherein said peptide has a sequence substantially the same as the amino acid sequence TKDNNLLGRFELSG (SEQ ID NO: 5).

7. The method of claim 1, wherein said activator is administered in conjunction with an interleukin.

8. The method of claim 7, wherein said interleukin comprises interleukin IL-2 and/or IL-15.

9. The method claim 1, wherein said activator is administered in conjunction with an inducer of the expression of said ligand on the cells' surface.

10. The method of claim 9, wherein said inducer is a histone deacetylase inhibitor.

11. The method of claim 10, wherein said histone deacetylase inhibitor is trichostatin A or suberoylanilide hydroxyamic acid (SAHA).

12. A method for inducing and/or enhancing cytolytic attack of NK cells against undesired cells in a subject which comprises administering to the subject a ligand for NK cell receptor NKG2D, a nucleic acid encoding said ligand, or an inducer of the expression of said ligand.

13. The method of claim 12, wherein further comprising administering an agent for inducing or enhancing the expression of HSP70 on the surface of said undesired cells.

14. The method of claim 13, wherein further comprising administering at least one compound which enhances an immune response or is administered in conjunction with such a compound.

15. The method of claim 12, wherein said undesired cells are tumor cells or infected cells.

16. The method of claim 12, wherein said ligand, nucleic acid or inducer is administered prior to, during or after exposure of undesired cells to an activator of NK cells.

17. The method of claim 1, wherein said cells are characterized by substantially lacking expression of HSP70 on the cells' surface.

18. The method of claim 1, wherein, said cells are induced to express HSP70 on the cells' surface.

19. The method of claim 18, wherein expression of HSP70 is induced by hyperthermia, chemotherapy, radiotherapy or any combination thereof.

20. A preparation comprising a ligand for NK cell receptor NKG2D, a nucleic acid molecule encoding said ligand, or an inducer of expression of said ligand in combination with an activator of NK cells and/or an agent capable of inducing expression of HSP70 on a cell's surface.
